(19) United States
(12) Patent Application Publication
(10) Pub. No.: US 2012/0136403 A1
(43) Pub. Date: May 31, 2012

(54) MEDICALLY PERPETUAL PERSISTENCE OF ESSENTIAL HUMAN LIFE

(76) Inventor: Spandan Choudury, Thane (IN)

(21) Appl. No.: 12/960,464

(22) Filed: Dec. 4, 2010

Related U.S. Application Data
(63) Continuation of application No. PCT/IB2009/052355, filed on Jun. 3, 2009.
(60) Provisional application No. 61/058,253, filed on Jun. 3, 2008.

(51) Int. Cl. A61N 1/36

(52) U.S. Cl. 607/2

(57) ABSTRACT

This Patent Application presents a biomedical mechanism of feasibly effecting tangible human immortality. The applicant believes that this technique is the only feasible and meaningful means of effecting perpetual persistence of human life. It forgoes all modern day approaches in the world up to now that target medical mechanisms to extend the life span of the body, and instead adopts a fundamentally novel innovation by way of enabling the biomedical transfer of the cognition that essentially constitutes a human to a different body prior to the death of the original body, whereby the transfer is NOT a copy of (and hence what would otherwise NOT be) the essential original human but IS the essential original human itself.

### CROSS REFERENCE TO RELATED APPLICATION

This continuation patent application claims the benefit of PCT International Application No. PCT/IB2009/052355 filed with RO/IB on 3 Jun. 2009 which is incorporated herein by reference in its entirety. (The aforementioned PCT International Application No. PCT/IB2009/052355 already claims the benefit of U.S. Provisional Patent Application No. 61/050,253, filed on 3 Jun. 2008 which is incorporated therein by reference in its entirety).

(Optionally, this patent application may instead be processed by the patent, office as a continuation-in-part patent application claiming the benefit of the same aforementioned PCT International Application No. PCT/IB2009/052355 filed with RO/IB on 3 Jun. 2009 incorporated by reference in its entirety—that continuation-in-part processing option for this application is however only being hereby made available to the patent office at a significantly lower preference than for the aforementioned continuation application option. See Claim(s) on why this application is lawfully no less eligible to be processed as a continuation application than it is as a continuation-in-part application).

### FIELD OF INVENTION

The field of invention is biomedical engineering. It primarily, but not only, involves neuroscience and electrical engineering. A novel, and possibly the ONLY feasible, means of effective persistence of human life for perpetuity is presented.

### BACKGROUND OF INVENTION

As indicated above, the applicant deems it futile of anyone to attempt to keep the human body alive perpetually—THAT WILL NEVER WORK. EVER, although it will be possible to increase the relative duration of the longevity of the body. It is a reasonable expectation that regardless of the state of research even millions of years from now, each body WILL die some time, either from old age, or external events (including diseases caused by external events, accidents etc). That, is because given the virtually unlimited count of interacting variables involved inside and outside a body to keep it alive, it will forever be impossible to successfully manage them all for eternity.

This Patent Application presents the applicant's novel invention on a practical mechanism of essential persistence of life for perpetuity.

The applicant sees two perspectives to human immortality—
A. Immortality only from the perspective of people that knew the original human, but not from the perspective of the original human.
B. Genuine immortality from the perspective of the original human.

The applicant infers that while it will in principle be technically feasible soon to transfer the cognitive and sufficiently important individual-defining non-cognitive contents of the human brain to a new brain (or another equivalent entity) whereby the people who knew the dead human would perceive the new entity containing the transferred data as essentially having simulated the original human, the key flaw in that approach is that the original human being will NOT feel as having continued life in the new entity—the original human's sense of self identity “I” would not be transferred into the new entity. Instead the second human's sense of self identity would be of a new “I” that would be a reasonable COPY of the original human's identity, and there could in principle be an unlimited count of such copies across multiple entities. That, however, does not constitute genuine immortality. In short, if the original human was “Mary” or “James” the second entity would still say “I am Mary” or “I am James”, but the original Mary or James would not accept the respective second entities as them, both when the original Mary or James are still alive and after their body-death. For purposes of this Patent Application, the original human will be referred to as “Mary-I”, while the second human per “A” would be “Mary-II-A”, and that per “B” “Mary-II-B”. This Patent Application pertains to the creation of Mary-II-B.

### SUMMARY OF INVENTION

This Patent Application is on the second perspective (“B.”) listed in the previous section, whereby the original human Mary-I would actually recognize herself as having been transferred into the new entity, with that entity being preferably a regular human brain attached to a regular human body.

There will be a common period of time prior to Mary-I's body-death when Mary-I and Mary-II-B would both be simultaneously alive, and while still alive Mary-I will be able to confirm her being able to recognize the (foreign) memory and cognition of the second brain and sense input from the second body. Mary-I will in effect be able to use not only her own brain's resources (i.e. her brain hardware and data) and input from the senses of her own body, but also be able to use the resources (i.e. brain hardware and data) of the second brain and receive input from the senses of the second body. Then, per the mechanism described in this Patent Application, Mary-I will willfully “transfer” her memory and consciousness to the second body. Of course, the people around the original human (Mary-I) would deem the second human (Mary-II-B) as essentially being even more of Mary-I, than would be the case with Mary-II-A.

### SUMMARY OF DIAGRAMS

FIG. 1—Demonstrates a sample neural network of 8 brains, including Mary-I's original, that forms an integrated, single, logical, augmented center of consciousness. As indicated, upon subsequent physical delinking, ordinarily at most only one of the 7 other brains can manifest Mary-II-B, while each of the remaining 6 can at most manifest Mary-II-A. Arrows indicate direction of flow of information, hence the direction of consciousness transport prospect, albeit the invention recommends dual-directional rather than unidirectional arrows in each case. Of course, the at-most-single Mary-II-B brain is only in as much as that only a single, logical, (augmented or diminished) integrated center of consciousness can be created to control one brain or a set of linked brains—and so it is optionally possible to have Mary-II-B to be created across multiple brains that are combined to form a single, integrated, logical center of consciousness. There will still ever be only ONE Mary-II-B, regardless of whether or not she is created in one brain or across multiple brains. The latter would not be recommended because of the logistical inconvenience that would be associated with the need to have multiple brains constantly communicate towards maintaining that logical, integrated center of consciousness.

FIG. 2—Demonstrates a sample neural network of 8 brains, including Mary-I's original, that forms an integrated, single, logical, augmented center of consciousness, whereby, upon subsequent physical delinking, while only one (ordinarily the maximum count permissible per this invention's inference) of the 7 other brains manifest Mary-II-B, only 3 of those 7 other brains manifest Mary-II-A and the remaining choose to retain their respective original identities. Arrows indicate direction of flow of information, hence the direction of consciousness transport prospect, albeit the invention recommends dual-directional rather than unidirectional arrows in each case. Again, the at-most-single Mary-II-B brain is only in as much as that only a single, logical, (augmented or diminished) integrated center of consciousness can be created to control one brain or a set of linked brains—and so it optionally is possible to have Mary-II-B to be created across multiple brains that are combined to form a single, integrated, logical center of consciousness. There will still ever be only ONE Mary-II-B, regardless of whether or not she is created in one brain or across multiple brains. The latter would not be recommended because of the logistical inconvenience that would be associated with the need to have multiple brains constantly communicate towards maintaining that logical, integrated center of consciousness.

FIG. 3—Part of hippocampal circuit pertinent to IBC

FIG. 4—Part of Papez circuit pertinent to IBC

For descriptive legends on one or more of the figures see subsection “Descriptive legends on figures” towards the end of the section “DESCRIPTION OF INVENTION”

### DESCRIPTION OF INVENTION

What is self identity and how is that relevant to perpetual persistence of life?

In the context of this invention it would be relevant to understand precisely what constitutes one's being aware of oneself and hence one's existence. While the views presented in this section are entirely the applicant's own, they do NOT by themselves constitute the invention itself, but instead justify/prove/validate the invention.

The first step towards that understanding would be to analyze if a human brain soon after conception were in principle medically isolated from all possible forms of input (to the brain) for decades afterwards while still being artificially provided all forms of nutrition to keep it medically alive and cause it to physiologically mature as brains would normally grow would at all have a consciousness and consequently have a sense of self-awareness. In other words, could a human brain develop consciousness when right from its inception it ceased to have absolutely any auditory, visual, olfactory, taste or tactile or absolutely any other form of input—the latter including a sense of pain, pressure, vibration etc. (which essentially are appropriate combined or singular renditions of the fundamental five senses anyway)? Regardless of whether that brain would still have medically “awake” and “sleep” cycles, including cycles of dreams, what could that brain base its consciousness on?

To answer that, one could ask oneself how one recognizes oneself cognitively immediately upon waking up from sleep. That awareness is from independent, corroborating recent (and sometimes past) memories of incidences, in addition to corroborating new input from the five senses with memories already in the brain of similar prior input (e.g. the visual/auditory input on surroundings being either similar to or different from what is already in memory, etc). Hence there need be some memory for self awareness. Memory is accepted as valid memory (and not imagination) with a high degree of conviction when it can be corroborated with other memory—and therefore the reality of memory is that each experience typically gets laid down in long term memory as a set of independent memories that effectively corroborate (and therefore reinforce/validate) each other—e.g., the memory of having simply sat in a chair somewhere could be recalled from various separate sitting-/time-/surroundings-/etc. angles, hence when recalling that experience one would essentially corroborate across multiple memories of that experience and therefore reasonably assess that one did indeed sit on that chair. In contrast to that, if all one recalled from that experience was just One Single Static memory of sitting on the chair, one would fail to adequately corroborate that memory with other memories of the same experience, and hence one would reasonably question the accuracy of that memory. The fact that most incidences are perceived across a span of time, however short, enables a memory of each such incident to be laid down as a set of corroborating memories, and of course the more intense an experience the greater would be the information content and count of such independent, corroborating memories. It could be noted that the count of memories of any experience would also increase with the intensity of that experience, even if the core incident behind that experience was fleeting. That is because the intensity of the experience would enable the individual to accept input (to be mentally tagged to that experience) on that experience for an appropriate period even after its occurrence. For example, if a person accidentally suffers a cut to a finger from a very momentary lapse of concentration while, say, cutting a piece of fruit to eat, the consequences of that cut would substantially add to the memory content of that experience, which would all be appropriately considered later for independent mental lookup and corroboration towards ascertaining whether one did indeed cut one's finger. As the brain continually lays new memories appropriate to the perceived significance of an experience, the longer an experience persists, and the more significant an experience is assessed to be at and shortly following the time it occurs, the more separate (yet linked, as they relate to the same event/experience) memories of it would be laid down that can later be collectively accessed for a corroborative validation on if the recalled past event/experience's having actually occurred. Each of those memories is built with various layers of the “knowledge framework of rules” (with basic rules at the lowest layers, and at the upper layers are more complex rules created using the relatively lower layers' basic rules and other rules of typical complexity not more that itself) that one gradually first develops at early childhood and continues developing on subsequently for one's entire lifetime (forming more and more complex rules). As particular complex rules are used more often, they themselves get directly interpreted to major extents without requiring referring the lower rules for interpretation, therefore increasing the speed of processing of related complex experiences. In short, self awareness requires memory, and for memory to form there have to be experiences, which are impossible in the absence of external input. It is important to recognize that if there is even a tiny amount of any input given to the brain at any time (e.g. in the case of any individual who might be born regular, but soon looses all physical senses within a day of birth), the brain would take that minimum information that was available for a day and naturally proceed to build up on that towards continually generating newer information databases—and in that case the brain WOULD be conscious. That build up of new information in that stated particular brain would largely be abstract of course as additional external input would be absent that would otherwise have enabled the buildup of a framework of a pool of rules adequately reflective of “reality”, but the brain would still develop an altered consciousness “in a world of its own”. It is significant to note that that state of abstraction is NOT truly unreal per se, but it is abstract in the context of the “real” world that regular humans live in. The human brain is designed to accept external input, however minimal, and, based on its significance, appropriately process the information contained in that input towards not only optimizing the subsequent application of the perceived rule(s) contained in that input, but also develop more complex rules premised on the basic rule(s) as voluntary and deemed necessary choices in anticipation of a need to apply such complex rules.

Therefore in the complete absence of all forms of external input, the applicant infers that it is logical that that brain would fail to have a meaningful form of consciousness.

There would essentially be no meaningful functional difference between its states of wakefulness and sleep, even if the brain goes through biological sleep and wakefulness cycles anyway.

The above conclusion is based on the hypothetical premise of a complete absence of external input into the neurons of the brain since the neurons' conception. In reality, that scenario is virtually impossible, because at the least the neurons Would be able to sense medical variations of the state of the brain itself as “internal” input that would be processed and developed upon towards an abstract perception of consciousness. In fact, even artificially rendering permanent unconsciousness to the brain since inception wouldn't make possible that state devoid of all input, since as long as the neurons are alive they would still automatically collect information from its surroundings, including, from within the brain itself, as well as from within itself. Of course the quality of consciousness would vary depending on the quality of input that it is based on.

However, the above conclusion is relevant to establishing the fact that being aware of the self and therefore being aware that one is alive mandates that the brain receive external input over a period at some point (at least in the past), as is enumerated further below. The mind of a regular human associates its sense of “I” with a virtually limitless count of memories that it can validate each of either entirely from other memories already in the brain related to the same event/entity/experience and/or with new input from the five senses. For example, one of those associations with one's self identity could be—“I recall having placed my finger on a cube of ice in the freezer compartment of my kitchen refrigerator a week ago, and my finger felt very cold” and “I recall placing my hand on a block of ice in the supermarket ten years ago and my finger did feel cold” and “Let me go touch snow ice that has collected outdoors in my garden this winter morning and let me try to experience if it indeed feels cold to the touch”. Each of those two memories correctly corroborate with each other affirming the individual's belief in the validity of those memories and therefore in the individual's identity of self. This is further reinforced by subsequently determining that snow ice feels the same to the touch. When the brain is totally relaxed, yet awake—that bandwidth of the sense of “I” tends to narrow (in the sense that there's a lower count of memories of the self active uppermost in the mind). When there's total amnesia of past memories and a complete loss of the ability to lay down new long term memories, one loses one's sense of identity completely, yet one can still function for basic tasks with short term memory (which do not contribute to one's self identity). A further degeneration of brain abilities would ultimately lead towards a gradual to a complete loss of even the ability to function.

A highly relaxed, awake human mind is of course not always entirely aware of its identity at any of the uppermost predominant layers of consciousness. Such periods typically are no longer than a short few seconds. That state is different from when the mind is so focused in other deliberations that one's awareness of one's own identity ceases to be a priority consideration for the duration of that focus, which also typically is no more than a few seconds. However, in a matter of seconds, in either case, one's identity comes actively back in one's mind at the appropriate upper level(s) of awareness. Of course since the levels of awareness in one's mind are made up of various layers consciousness (most conscious at the highest and least conscious at the lowest), self awareness is manifested only in the appropriate layers, commensurate with the significance of the need to be self aware (per the ongoing deliberations in one's mind, and where relevant the external input as well). It is to be noted that even while one might not be consciously aware of the more profound aspects of one's identity during the stated periods of intense alternative focus, one still retains a form of self awareness in more simple parameters—by comparing incoming external input with short- and long term memory of the same input. For example, that awareness continuity could be in the form of a breeze blowing across one's face before and then, or some background sound persistent before and then, et al.

Therefore, what a human cares for in as much as being alive is to be able to CONSCIOUSLY refer back—and therefore naturally corroborate across—as many of those memories as is possible, in the process not only consciously and subconsciously affirming the individual's self identity but laying new memories (in the form of plans, etc) towards feeling positive about one's self identity in the long run on the whole. The latter is of course achieved in a complex combination of learnt behaviors (outside the scope of this Patent Application) that ultimately release the “feel good” neurotransmitter et al chemicals in the brain (e.g.

(A). learning that society currently affirms that one must love and take good care of one's child and therefore one must do good to one's child, based on which genuine love for the child develops (albeit that love also develops in the absence of society based on other aspects related to memory in the human mind), therefore doing good to one's child or seeing the child happy releases those feel-good chemicals in the brain.

(B). learning that society encourages success, hence planning for and attaining success releases those feel-good chemicals in the brain.

Therefore, that self identity (and not the brain hardware associated with it) is what constitutes being a particular human, and it is that self identity that the invention described in this Patent Application focuses on perpetually transferring sequentially, towards life's persistence in perpetuity. To be noted is that brain hardware is of course critical for that identity processing to be done, and subtle variations in the hardware of the brain would cause subtle changes in the perception of one's self-identity even with everything else remaining the same—for example if a pair of (genetically identical) clones were kept in the same environment since inception, they would NOT develop to have the exact identical memory banks or personality. However, the applicant infers that most brains' hardware is acceptably identical within reasonable limits in the context of this processing (distinguishing a human brain from, say, a dog's brain or—as an extreme incompatible example—a latest modern day supercomputer), rendering it possible that most brains be acceptably compatible to process that transferred self-identity in as much as their being able to reasonably manifest the original human's self awareness and traits.

For feasibility, this invention uses the brain's existing abilities and properties towards that transfer, as elaborated subsequently in this Patent Application.

The quality of human consciousness containing (by definition) one's self identity, even for the same individual, is plastic. There are multiple, non-discrete qualitative levels of increasing or decreasing consciousness. In all cases when one can reasonably identify oneself, one ultimately normally has a positive experience with one's being alive and awake, hence it would be any of these states of consciousness (where one can meaningfully identify oneself) that would need to be transferred in the context of immortality. However, when there is no sense of self identity at all (when, for example, there's total amnesia of past events associated with a complete inability to lay any new memory) even while the ability to otherwise fully function with short term memory alone is retained, the state would not be meaningful to transfer towards immortality because there is no self identity in such a case.

### Key Feasibility Controls

At the core of this invention are the following controls that render this invention feasible—

(i.) The applicant's inference that what constitutes being any particular human is Not tied to any one physical brain, but the awareness of memory content and new external input towards direct and indirect self-recognition (i.e. consciousness of the self) and learned reactive rules (i.e. personality, which to a major extent is essentially a manifestation of consciousness of the self) (more importantly the former, because human personality can be modified/learnt later but critical information needs to be retained if self-identifying memories have to be passed on) that can each be Consciously (and Otherwise, primarily the former)—While the Original Brain is Alive—Rendered Familiar with a New Brain's Hardware and its Information Content, Integrated for Functioning (While Still Keeping the Original Information Separately Identifiable) with the New Information Content and Hardware of the Second Brain Along with the Original Brain Hardware. This Process can be Extended to Beyond a Set of Just Two Brains. Consequent to that and the other controls listed in this section the Applicant's Inference that any One Original Brain is able to Consciously (and Otherwise, primarily the former), Following the Stated Integration with One or More Additional Brains (& the Information Contained Therein), Choose One of Those Brains to Base its Consciousness (and therefore personality too) On. That is Based on the Fact that Each Individual Human Brain Constitutes a Reasonable Minimal Hardware that is able to Support Adequate Human Consciousness by Itself, Albeit the Quality of that Consciousness is Expected to be Slightly Inferior to the Integrated Consciousness Attained with Multiple Brains.

(ii.) The applicant's inference that there cannot be more than one integrated center of consciousness (that, by definition, acknowledges complete self identity) for any living entity at any one time. There can be multiple separate integrated centers of consciousness, each of which could in principle separately take complete control of one's entire core consciousness at different points of time, but only one can he the sole integrated center of consciousness at any one time. To rephrase, There can be at Most be One Logical (and hence PHYSICAL (which crucially doesn't entirely HAVE to be at one location—just that the constituent physical components need to not be physically delinked)) Center of Consciousness for any Living Entity at any One Point of Time. To rephrase, in the context of the human, when a set of integrated brains (manifesting an integrated center of consciousness) are physically delinked, the consciousness of that set can at best be transferred to AT MOST one human brain's center of consciousness (or—if chosen—an integrated, augmented logical center of consciousness formed of interlinked centers of consciousness of multiple regular brains) (and at worst to no human brain at all—the integrated consciousness, would just be destroyed), in as much as a human brain is considered the minimum (but NOT the maximum) physical hardware capable of adequately hosting a comprehensive center of human consciousness that (i.e. the consciousness) can be fully independent of reliance on any other hardware (i.e. any other brain), as is the case with every known regular human brain today. This for example, is to contrast the regular human brain from that of a primate in much as the latter being incapable of physically hosting a human-quality center of consciousness. Again, that is not to state that consciousness WILL be transferred to one brain even if not desired, but only that AT MOST one center of consciousness (spanning one or multiple brains) can have the integrated consciousness transferred to (if applicable/necessary at a slightly inferior quality, but adequate and comparable to the quality of consciousness in any regular human brain). Again, it is important to note that in principle it is 100% possible that if so elected an integrated center of consciousness can be transferred to span across multiple human brains whereby the multiple brains would either together host a SINGLE integrated center of consciousness or independently host separate centers of consciousness only one of which would take full control at any one point of time, however such would render this invention less practicable and less useful. because of the then need for perpetual communication and cooperation among multiple brains that typically would also imply the requirement of the same count of independent, perpetually collaborating humans.

(iii.) The already publicly recognized ability of the brain to attempt to functionally optimally (i.e. as best as is possible given the tissues and other aspects of the scenario) communicate with neurons not part of that brain, and with the quality of communication augmented in a technology enabled environment. This has been independently evidenced in various attempted-regenerative/“curative” procedures involving the cerebrospinal system.

(iv.) The brain's already publicly recognized ability to still function optimally (as best as is possible) without the original entirety of the brain, on account of information content multi-duplication and redundancy across the brain, brain hardware redundancy and brain hardware function flexibility. This is evidenced in cases of traumatic brain injury or surgical removal of brain components.

(v.) The (human) brain's already recognized ability to learn to do just about anything, with and/or without conscious will. There is ho dearth of evidence on the brain's remarkable learning abilities.

### Specific Steps in Invention

### Step 1—Link Brains to Communicate

[Key Feasibility Control Applied—(iii.)] (Ref. “Key Feasibility Controls”)

Mary-I is to be linked to the new brain.

The intention is that subsequently, per Step 2, Mary-I's brain naturally integrates (enabled by appropriate, minimal external technology support (Ref. “Technology supported interbrain communication”)), as far as optimal logical functioning, with the brain of the second individual towards jointly forming an augmented logical neural network, essentially aimed at rendering the two brains capable of functioning as one, while still maintaining their separate identities at will.

In order to be meaningful (for example for protection from transmissible terminal illnesses like hepatitis, cancer tissue, HIV etc) this integration will Not mandate a direct tissue connection, but instead can even be achieved with wires, or even entirely wirelessly.

Step 2—Learn to Use Other Brain, Towards Forming (Together with Mary-I's Own Brain) an Integrated, (Virtual) Single, Augmented, Logical Brain Containing an Integrated, Augmented, Logical Center of Consciousness
[Key Feasibility Controls Applied—(i.), (ii.), (iii.), (v.)] (Ref. “Key Feasibility Controls”)

This is the most critical component of this invention, whereby each of the two connected brains would, using a combination of the appropriate, minimal external technology support and optimal mental effort by way of using the brains' natural ability to learn and optimally integrate with new hardware, gradually recognize the hardware and information content of the other brain, towards forming a single, augmented, logical brain with a single, augmented, integrated logical center of consciousness.

That single, augmented, integrated logical center of consciousness will include, in whole or in (typically the most) part, the logical center of consciousness of each individual brain, and (typically) an additional consciousness component originally present in neither of the two original centers of consciousness. That additional consciousness component may preferably be reduced or eliminated for the integrated, augmented consciousness to maintain acceptable fidelity to the logical integration of the original set of consciousnesses.

The aim of this step is to establish one logical center of consciousness (while still consciously and otherwise retaining the separate identities) for the combined brain at any one point of time, such that it can be switched on or off at will (by the combined single logical center of consciousness, with (importantly—so that the rights and will of each original human are never compromised at any point) each original separate logical center of consciousness retaining full overriding control on its own share of original physical resources and original information content) to return back to the state of the two separate logical centers of consciousness. In short, the combined logical center of consciousness will happen only if both original separate centers of consciousness agree, while never losing awareness of and control on their separate original identities (i.e. on the separate original logical centers of consciousness), and should one of those identities wish to exit the combo it could. The combo would essentially remain an optimally integrated sum of two separately distinguishable parts that could choose to either separate or work together—with each maintaining the critical ability to recognize and influence the other. So there is an element of trust involved, necessitating a correct selection of the two original human candidates, and it would be meaningful that with that selection correctly done the logic of mutual- and overall good would largely prevent infraction of rights by the combined logical center of consciousness of its separate always identifiable component original centers of consciousness: in fact, it would also be easy for each brain's consciousness to willfully deny detrimental “takeover” against its will by another brain's consciousness, much though not exactly the same way for example the Autonomic Nervous System in any standalone individual optimally controls that individual's heart rate, digestion etc without direct conscious control by that individual's consciousness albeit the individual would still be appropriately aware of the individual's heart rate, digestion, etc. In other words, the consciousness combo in the combined logical center of consciousness would remain appropriately aware of each component center of consciousness and be able to control that component center of consciousness to an extent appropriately less than the extent of its awareness of it. As an additional option, that awareness by the integrated center of consciousness of each component center of consciousness and consequently of the human body originally attached to each respective component center of consciousness can be but does not have to be 100%—in other words it could be but doesn't have to be that sections of a component center of consciousness and consequently of the original human body attached to that component center of consciousness remain completely out of bounds of the integrated center of consciousness, not just in Step 2 but even at the conclusion of Step 3. That one logical center of consciousness could be physically located at either brain, or in both combined—of course ideally the former for portability logistics so that Mary-I's original brain hardware could be completely disregarded at will (if so chosen by the stated combined single logical center of consciousness, (pre-) approved by Mary-I).

After the two brains have been linked to communicate so that the process of logical functional integration could begin, the next step would be for Mary-I to consciously (and sub-/unconsciously) familiarize with the second brain's hardware's peculiarities and with the information content therein, and consequently “learn” to cogitate using “both” her existing brain as well as the second brain's hardware resources and information content. This process could last several hours up to multiple days (in sessions), in the earlier phases of implementation of this invention, and would expectedly very significantly speed up with improvements in supporting technology later. The stated processes of logical integration and learning by both the two brains would proceed at the same time. The neurons of the brains would know how to optimally integrate and function jointly, as long as a potent communication environment (outlined separately in this Patent Application) is provided. At the end of the process, the brains would be deemed to have provisionally been functionally integrated towards optimal logical operations as one (virtual) single, augmented brain. That virtual single brain would have two separate physical centers of consciousness of course (that would, at mutual consensus as part of the process of learning, switch off or on alternately and simultaneously as part of that learning), and Mary-I's brain and the other brain would each learn to recognize the logical consciousness center of the other brain, and as part of the familiarization process grant alternating permission for trial control of consciousness and data (i.e. retrieving and adding memory, processing new input from body etc.) by the other brain's center of consciousness. Essentially Mary-I's sense of self identity would now include recognizing new data and brain behavior that were not originally hers and which she would reasonably “tag” as such (i.e. she would not confuse new information in the second brain with her own original information in her brain, instead she would tag new information as not originally hers and simply learn to deal with that information while maintaining her identity in the integrated logical combo). As part of the integration, she would also learn to recognize and work with the other brain's different physical components and data therein towards manifesting her own original personality, to the extent that her original personality would need to be reasonably exhibited at will following the transfer. While a significant portion of the learning will be in conscious states, part of the learning will be in sub-/unconscious states as well. This process of “learning” towards a logically integrated single (virtual) brain with a single (virtual) logically integrated center of consciousness made up of the always identifiable separate original two logical centers of consciousness (with two physical centers of consciousness that will not always be either simultaneously awake or simultaneously asleep, by mutual consensus) where each brain learns to recognize and work with the other brain's physical components and data therein towards manifesting one's original self-identity and personality and other adequately significant aspects of oneself using one's own and the other brain's hardware, and identifies and tags the other brain's information content as not being one's own original memory, and with the combined single logical center of consciousness maintaining the ability to be switched on or off at will (by the combined single logical center of consciousness itself (or by one/more individual component original logical center(s) of consciousness where applicable), with (importantly—so that the rights and will of each original human are never compromised at any point) each original separate logical center of consciousness retaining full overriding control on its own share of original physical resources and original information content) to therefore enable a return to the original state of two separate logical centers of consciousness (if so desired), is the first significant step of this process. Post transfer, the traits of either Mary-II-B or the original second individual would be manifested at will whenever chosen. In effect, the “better” traits would ordinarily manifest (again, largely at will)—for example if Mary-I was level headed and the second individual originally was less so, then the combo would be level headed at will, and if Mary-I was poor in Math while the second individual was not then the combo would not have to be poor in Math.

The centromedian nucleus, which is part of the ILN (Intra-Laminar Nucleus) of the thalamus, is already recognized in the medical community today as an important center of consciousness in the human brain. Since, as part of the “learning” phase, the two separate brains would need aim to function as one with preferably hut not necessarily one physical center of consciousness (towards attaining the post-learning-completed requirement of having one logical center of consciousness for the combo at any one point of time, albeit with the combo's never losing awareness of the two separate identities), the applicant recommends enabling neural networking that allows the two centromedian nuclei and other separate components that all jointly constitute the two integrated centers of consciousness in the two brains to both remain functional (to allow for both the independent brains to learn to recognize and “learn” each other and consequently enable both operate jointly as one center of consciousness and also separately as two separate centers of consciousness that each brain would be able to sense both of), as well as alternatively temporarily disabling the integrated center of consciousness in each brain. For part of the process, both brains' centers of consciousness would optionally need to be simultaneously disabled (naturally as sleep, and artificially with technology support) to allow natural, enhanced with technology support, optimized information dissemination from the other brain into each brain. During the process, information from the second brain would be naturally, enhanced with technology support, optimally integrated with Mary-I's brain, and vice versa, resulting in Mary-I's being able to consciously sense and deliberate with memories in the second brain and sense and process input from the second body. The second brain would be able to do the same with memory in Mary-I's brain and input from Mary-I's body.

Mary-I and the other individual could be geographically located separately anywhere (albeit in the earlier incidences of implementation of the mechanism in this Patent Application they should be in proximity), and at the conclusion of the process of that optimal functional integration, Mary-I and the second human would each have a voluntary (i.e. it can be switched on and off at will), augmented cognitive ability primarily Solely (i.e. in the most part) using the physical resources of the other brain. The centers of consciousness of the two separate brains would also be able to at will operate as one or separately. At that stage, either Mary-I or the other individual, could be relocated to the other brain.

The applicant emphasizes that in this invention dual-directional transfer is more recommended than unidirectional, towards a higher degree of potency of the process.

Step 3—Relocate to Second Brain and Physically Disassociate from Original Brain
[Key Feasibility Controls Applied—(i.), (ii.), (iii.), (iv.), (v.). The notable inclusion of (iv.) is relevant for reasons of the reduction (by way of disassociation from the original brain (hence from the center of consciousness therein)) of the augmented, integrated, logical brain (hence of the augmented, integrated, logical center of consciousness) of Step 2] (Ref. “Key feasibility controls”)

The sequence of the two operations in this Step 3 surmised in the step title above is important—relocation at will happens first, physical disassociation with the original brain happens next.

With the two brains having already been functionally integrated in Step 2 towards the creation of one logical, augmented, integrated single center of consciousness (that will also have, to appropriate extents, retained awareness of and control on the separate original identities), it would be necessary here to get the two brains' centers of consciousness suitably collaborating towards confirming with Mary-I that she can acceptably sense an integrated consciousness using the centers of consciousness and memory in both the brains and input from both the bodies and then checking that the same responses are consciously made representing Mary-I from the second individual. Next, Mary-I would consciously (and otherwise, with external technology support) actively “relocate” in a highly focused, deliberated and conscious fashion to the augmented, integrated, logical center of consciousness and voluntarily (by focused deliberation or otherwise, with external technology support) disable (or render less relevant) her own brain's center of consciousness thereby gradually relinquishing control of self identity from her own brain and allowing self identity to be processed from the augmented, integrated, logical center of consciousness (which by then would essentially comprise of, in whole or in (typically the most) part, the logical center of consciousness of the second brain, and (typically) an additional consciousness component (that, as indicated elsewhere in this Patent Application, may be reduced or eliminated) originally present in neither of the two original centers of consciousness) such that when her original brain is then disassociated her consciousness (and hence by definition her self-identity) would all the while have remained active in the other brain, she becoming Mary-II-B. Implementing the process while conscious per the critical procedural preference stated above would render the process reasonably safe in the sense that Mary-I would definitively know that she has transferred to the second brain prior to her death.

### Step 4—Repeat Steps 1 to 3 for Essential Immortality

Mary-II-B can repeat the three steps 1-3 at an appropriate time before the physical death of her new body. This cycle of 4 steps is continued, to render Mary-I effectively immortal.

### Other Considerations

To be noted here is that memory/personality information in the sections of the brain that are not relevant to conscious cognition (e.g. breathing, swallowing, etc) will be given a lower priority for transfer. The reason for that is that while those components do have a minor contribution to self identity, that contribution is relatively minimal. Most of these lesser significant to self-identity inadequately cognitive body functions would be directly taken up by the host body's already existing capabilities.

### Fair Scope of Invention

Without limiting itself to (i.e. irrespective of) any specific support hardware, this Patent Application covers the four-step process earlier detailed, even when more than two brains are involved. The latter is possible, per discussions in this Patent Application, if for example Mary-I chooses to link to multiple (and not just one) other brains towards forming an integrated brain with an integrated consciousness center, prior to delinking from them all into one brain (or—not recommended—multiple brains, towards an integrated, augmented center of consciousness) of her choice to form Mary-II-B.

In short, this Patent Application encompasses not only all morphs/variations/renditions/forms/flavors of the combination of the three steps (Step 1, Step 2 and Step 3) with and without Step 4—enumerated in the section “Specific steps in invention”—but also (all morphs/variations/renditions/forms/flavors) of the following core sequence of three steps Step A, Step B and Step C, with and without Step D (that together constitute an adequate, accurate superset to the steps in “Specific steps in invention”), in the premise of, but not limited by, the applicant's comprehensive logic, details, options and variations elaborated in the entirety of this Patent Application. As is self evident, all applications of existing or future technologies in the context of all morphs/variations/renditions/forms/flavors of the sequence of three steps—Step A, Step B and Step C—with and without Step D, are included in the scope of this Patent Application. Notably, Step B and Step C by themselves or together in any sequence, whether or not preceded by Step A and succeeded by Step D are also included in the scope of this Patent Application. Step A by itself is covered in the Patent Application only to the extent that it is relevant to medically perpetual persistence of essential human life as detailed in this Patent Application, as well as to the form of connection outlined in (and therefore may be lawfully expanded or extrapolated on as being included in this Patent Application's scope) Step 1 of “Specific steps in invention”. Step A and elsewhere in this Patent Application and to the application of interbrain communicating technologies described in “Technology supported interbrain communication” and elsewhere in this Patent Application. In extension, of “Specific steps in invention” Step 2 and Step 3 by themselves or together in any sequence, whether or not preceded by Step 1 and succeeded by Step 4 are also included in the scope of this Patent Application. Step 1 by itself is covered only to the extent that it is relevant to medically perpetual persistence of essential human life as detailed in this Patent Application as well as to the form of connection outlined in (and therefore may be lawfully expanded or extrapolated on as being included in this Patent Application's scope) that Step 1 of “Specific steps in invention” and elsewhere in this Patent Application and to the application of interbrain communicating technologies described in “Technology supported interbrain communication” and elsewhere in this Patent Application. However the technologies themselves, to the extent not discussed in this Patent Application and to the extent not applied towards all morphs/variations/renditions/forms/flavors of one or more of the three steps below (Step A [except when Step A is excluded from the Patent Application's scope as described above]. Step B and Step C, in any sequence, with or without Step D) would be outside the scope of this Patent Application. Again, the novelty and hence the scope of this Patent Application is not limited to the application of the supporting technologies towards the steps below and (application) to the steps in “Specific steps in invention”, but importantly includes the steps themselves. The novelty of the steps is MORE significant than the novelty of the application of the technologies themselves towards manifesting those steps. However, the fact that the invention entails the use of technology as a necessary aspect of each of the four steps contributes to enabling the invention's lawful patentability.

A. Mary-I's brain is linked to one other brain, or multiple other brains.
[Key Feasibility Control Applied, Appropriately Extrapolated—(iii.)] (Ref. “Key Feasibility Controls”)
B. Mary-I learns (using a combination of (various levels of) conscious-, various levels of subconscious-, and (various levels of) unconscious-initiatives with technology support) to use other brain(s), towards forming one integrated, augmented brain (or a combination of communicating integrated, augmented brain systems) containing one integrated, augmented, logical center of consciousness (or a combination of communicating, integrated, augmented, logical centers of consciousness, only one of which would have authority over the set of participating brains in that particular augmented, integrated, logical center of consciousness, at any one time). Each integrated, augmented, logical center of consciousness would comprise of in whole or in (typically the most) part, the logical center of consciousness (hence include an appropriate fraction of the consciousness component therein of the associated brain) of each individual constituent brain, and (typically) an additional minor consciousness component (that may preferably be reduced or eliminated for the integrated, augmented consciousness to maintain acceptable fidelity to the logical integration of the original set of consciousnesses) originally associated with none of the constituent brains. Each integrated, augmented, logical center of consciousness will be physically located across one or up to all of the individual single original centers of consciousness in each constituent brains—preferably (for upcoming (per Step C) Mary-II-B's logistical convenience) in exactly one of the constituent brains' centers of consciousness—and only one integrated, augmented, logical center of consciousness may control a constituent brain at any one point of time, even though that brain may be part of multiple separate integrated, augmented, logical centers of consciousness. As part of that learning Mary-I would consciously, subconsciously and unconsciously adequately recognize herself (inclusive of all, most or an acceptable extent of her key original memories) using the (and each, when multiple) integrated, augmented, logical center(s) of consciousness—hence the logical center(s) of consciousness in the other brain(s). That recognition would alternatively be, at Mary-I's conscious/subconscious/unconscious initiative/will, using some/all the centers of consciousness (i.e. at will, including her original own and the other(s) and separately using only the other(s) and not her original own). As indicated at the top of “B”, at the end of this step will be formed one integrated, augmented brain (or a combination of communicating integrated, augmented brain systems) including Mary-I's brain, containing one integrated, augmented, logical center of consciousness (or a combination of communicating, integrated, augmented, logical centers of consciousness, only one of which would have authority over the set of participating brains in that particular augmented, integrated, logical center of consciousness, at any one time).
[Key Feasibility Controls Applied, Appropriately Extrapolated—(i.), (ii.), (iii.), (v.)] (Ref. “Key Feasibility Controls”)
C. Mary-I chooses a logical center of consciousness if multiple individual logical centers of consciousness have been created in step B., “relocates” to it, logically disassociates from Mary-I's original brain (i.e. from the original center of consciousness of Mary-I) (Note—while a physical disassociation from Mary-I's brain is not mandatory, that will typically ultimately follow), and therefore manifests Mary-II-B within that chosen logical center of consciousness. The logical center of consciousness would ideally be located physically in just one of those other (i.e. non-Mary-I) brains—hence in the center of consciousness therein that one brain—for enhanced logistical convenience of Mary-II-B (but could in principle be located in more than one of those brains as well, as long as they're logically linked forming one integrated, augmented, center of consciousness). At the and of that transfer Mary-I's brain will be logically, and in practice often physically, disassociated from the integrated, augmented, logical center of consciousness that Mary-II-B has relocated to.
[Key Feasibility Controls Applied, Appropriately Extrapolated—(i.), (ii.), (iii.), (iv.), (v.). The notable inclusion of (iv.) is relevant for reasons of the reduction (by way of disassociation from the original brain (hence from the center of consciousness therein)) of the augmented, integrated, logical brain (hence of the augmented, integrated, logical center of consciousness) of Step B] (Ref. “Key feasibility controls”)
D. For essential immortality of Mary-I, Mary-II-B will repeat steps A, B and C at an appropriate time prior to the physical death of Mary-II-B's (new) body. The cycle of 4 steps is repeated for as long as Mary-I is wished to be kept alive.

To clarify scope, while the manifestation of Mary-II-B is complexly covered within the scope of this Patent Application, the manifestation of Mary-II-A in itself is not covered in the scope of this Patent Application as long the scope of this Patent Application detailed in the rest of this section (“Fair scope of invention”) is otherwise rigorously applied—to rephrase, the use of morphs/variations/renditions/forms/flavors of the stated four-step process towards the creation of Mary-II-A is covered in this Patent Application's scope.

### Proof of Feasibility/Tangibility/Concreteness/Originality/Innovation, Hence Patentability

The sections “Key feasibility controls” and “What is self identity and how is that relevant to perpetual persistence of life?” outline the primary bases of feasibility. Additional feasibility considerations are in the sections “Recommendations”, “Deliberations & clarifications” and “Technology enabled interbrain communication”. “Specific steps in invention” and “Fair Scope of invention” make explicit application of the bases of feasibility surmised in “Key feasibility controls”.

The proof of tangibility is of course in the perpetual continuity of life this invention achieves.

The proof of concreteness is in the specific four steps listed in the sections “Fair Scope of invention” and “Specific steps in invention”.

The proof of originality and innovation is of course in the very fact that feasible, tangible and concrete perpetual persistence of essential human life constitutes this invention, because perpetual persistence of human life has never been possible yet in any other invention by humankind.

While the novelty of the overall four steps of the invention is substantially more significant than the novelty of the application of the technologies themselves as part of the invention towards manifesting those steps, the integration of such appropriate technologies as a necessary aspect of each of the four steps of the invention, therefore and otherwise rendering the overall invention original, innovative, feasible, tangible and concrete, contributes to enabling the invention's lawful patentability. Additional details on feasibility could be provided upon request—reflecting that it is appropriate to neither limit the applicable technologies nor immediately build a physical prototype because:
Many different technology options are currently feasible for this invention, with and/or without the relevant follow up insignificant (and/or optionally significant), time spanning, observation and analysis, whereby the immediate patentability of this invention remains entirely undiminished, and is in fact augmented, by such follow up—and other technical considerations.
A complete physical prototype would be feasible if sufficient funding, time and direct and/or at least indirect public will (for example, but not limited to, in terms of a commercial end user market for the invention) are confirmed.
The immediate building of a physical prototype is as unnecessary to affirming the validity (i.e. originality, novelty, feasibility, tangibility and concreteness)—and hence the patentability—of this invention as would be, for example, for any other entirely unrelated invention (not in the scope of this Patent Application) whose originality, novelty, feasibility, tangibility and concreteness are entirely self evident, yet building a physical prototype would require the availability of funds, time and/or at least indirect public will.

### Recommendations

It is important to note that as part of the applicant's considerations on quick implementation feasibility the applicant emphasizes on the following—
Recommends that consciousness be preferably transferred across existing brains of reasonable maturity. Of course the more similar the second brain is to Mary-I's original brain (if largely “regular”), the better would the quality of Mary-II-B be—and if Mary-I's original brain is less than regular (e.g. from a congenital- or injury induced hardware shortcoming) then the target brain to transfer to should ideally be regular. Of course the younger the second individual would be, generally the better, in order to increase the likelihood of minimizing the frequency of such transfers (including, but not limited to, by delaying the onset of normal “old age” post transfer). If inter-brain communication is aggressively tuned per the direction outlined in this Patent Application, the applicant infers that the creation of Mary-II-B will begin being possible any time within 8 or 10 years.
Includes the possibility of rendering consciousness transfer across clones, which in principle would be the ideal form of consciousness transfer. That is because the technology to create clones is available today, and it would be supportive of this invention to create a young Mary-II-B when Mary-I has already reached a relatively senior age.
Strictly excludes fancy considerations like transferring to a computer. That is not practical simply because the inherent hardware design of the modern day computer vastly reduces the likelihood of the hardware being able to sufficiently multi-process with extremely low consumption of energy to render the computer portable and otherwise feasible. In short, the modern day computer is completely counter to hosting cognition even remotely as optimally as human cognition in the human brain—the best modern day supercomputer does unacceptably more work than the human brain would for the same job.

### Deliberations & Clarifications

Important considerations in this invention are the following—
a. The same brain and body resources cannot be set to be simultaneously controlled by two separate logical consciousness centers. At most only one logical center of consciousness can take control at any one time. In extension, there can at one point be only one (integrated) logical (i.e. constituent components not physically delinked) center of consciousness as far as self identity is concerned. Refer “f.” below.
b. It is not necessary for every bit of information in Mary-I's brain to be transferred to the second human. That is because human memory and personality are both plastic and are changeable. As long as a reasonable amount of information is transferred that would enable Mary-II-B to acceptably recognize herself, that would constitute a good start. Innate human cognitive capabilities would then naturally do the needful to attempt to build up a new memory bank and personality using that information. With the passage of time as newer and faster information transfer techniques are developed, Mary-II-B's brain (excluding prior information already in the second brain) would more closely represent Mary-I's brain at the time of transfer.
c. The technology to clone any human organ is already available today. Therefore, if the center of consciousness (namely the centromedian nucleus and allied components) for any human brain were cloned and connected in parallel to the same endpoints in the rest of that specific brain (namely sending nerve fibers to the cerebral cortex, putamen, caudate, collateral fibers etc. and receiving nerve fibers from the spinothalamic tract, cerebral cortex, superior colliculus, reticular formation, globus pallidus, vestibular nuclei etc.), would the sense of “I” be adequately realized in the cloned center of consciousness such that the two parallel centers of consciousness could operate in the following ways?
1. As one combined augmented logical center of consciousness.
2. As two separate logical centers of consciousness where each center remains aware of the other center and complete control of the resources (and hence of one's consciousness, and hence, by definition, of one's self identity) by any one center at one time is taken by voluntary joint consensus.

The applicant infers that the answer to both is “Yes”.

As an extension, “1,” would be possible differently if the cloned center of consciousness were not just connected at the ends with the original but entirely merged with the original.
d. If, in addition to the center of consciousness, the rest of the entire brain were cloned too, such that initially each component still remained functionally part of the corresponding original brain such that the entire brain functioned as a “super” brain with one consciousness, and then if the two sets of components were separated to form two separate exactly similar brains, would the original single sense of consciousnesses be copied in both brains and if so how would it be in relation to the original single sense of identity?

Obviously, with no physical connection between the two brains, the hitherto similar manifestations of the same consciousness would thenceforth develop into two distinct flavors of consciousnesses in separate ways from the point of separation. The original sense of self identity could remain in any one of the two brains, but never in both the physically disconnected brains at the same time. To rephrase, Mary-II-B can be manifested in at most one of the two physically disassociated brains.
e. Mary-I should select the individual to transfer to well in advance of her anticipated death, and effect transfer of all meaningful long term memory in advance. Following that, Mary-I should periodically transfer further long term memory since accumulated. Ideally this whole process should be undertaken with at least two geographically apart individuals to minimize the risk or not ultimately having any individual available to transfer to just prior to Mary-I's death.
f. Could Mary-I be transferred to multiple third party brains? The applicant infers that the answer is “NO” as far as Mary-II-B, but ‘yes’ in the context if Mary-II-A. There HAS to be at most one single logical (i.e. constituent physical components not delinked) center of consciousness where one can acceptably-self-identifiably transfer to at any one point of time. Therefore, while it is possible for multiple brains to function together as a whole (as earlier detailed), there HAS to be only one logical center of consciousness for them all as far as acceptable self-identification. That integrated logical center of consciousness doesn't have to necessarily physically be entirely in any one of the linked multiple brains, but suitably distributed across them all—non-functional individually but functional as a whole. Therefore when the physical link between the brains is broken—the actual transfer (towards Mary-II-B) would AT BEST be in only one brain—the other physically delinked brains can AT BEST have COPIES (towards Mary-II-A, that, as earlier elaborated, would not be acceptably-self-identifiable) of that original identity where SEPARATE, independent development would proceed from that point of physical disassociation. AT WORST, the transfer wouldn't happen at all, and the original individuals would just be left with new memories that they'd recognize as not theirs (since the memories would not involve them, but primarily Mary-I, and secondarily some of the other people in the set). What would NOT HAPPEN is Mary-II-B's manifestation in more than one separate brains.
g. Could, following a transfer, Mary-I separately sense herself being present in both her original brain and in the second brain that is no longer connected to her own still alive brain? This is an extension of sub-query “d.” and “f.” above, and the answer is “No”—she can be transferred as Mary-II-B to another brain while still alive, as long as she volunteers to let her own brain's center of consciousness give up control of her consciousness to the second brain's center of consciousness.
h. In the context of a brain that has been devoid of all forms of external input to it since inception, would that brain have medically wake and sleep cycles, and if so would the brain have dreams in those sleep cycles? Extending upon the behavior of a human baby in the womb, it can be reasonably presumed that the brain probably would have sleep and wakefulness cycles, simply because that property appears to be genetically so entrenched in the system that it would manifest itself regardless of input. As far as dreams—the applicant infers that an entirely-input-devoid brain does not logically appear to be capable of any cogitation because there would be no basic rules framework in the brain's memory database that can be used to cogitate with—and since cognition appears to be logically impossible, dreams in the way we know it appear to be impossible as well. In other words, there would potentially be no meaningful difference between that brain's state of wakefulness and sleep/dreams. However, this inference is not directly related to this invention.

### Technology Enabled Interbrain Communication

The applicant infers that it is feasible to have two separate brains (learn to) communicate ((at least) initially with active conscious will, to enable the brains learn to recognize the signals and hence the communications mechanism) in a micro-focused electromagnetic communications environment of multiple simultaneous signals of the appropriate strengths and frequencies focused at equivalent brain components in the two brains, as briefly expounded in the following paragraph. Although neurotransmitters could potentially be used to render the process more potent, the applicant deems that essentially unnecessary given that the electromagnetic signals received would anyway enable the natural generation of the appropriate neurotransmitters as the brains deem necessary to process those signals. Essentially, the detected output signals from the relevant components of one brain would need to be appropriately modulated and micro-focused on the appropriate input components of the other brain. That modulated input would be in the form of ultra-low voltage signals at compatible frequencies and strengths (and duration for transfer effectiveness (because transfer will not always be real time, but suitably time lagged mandated by the complexity (i.e. relevant ranges of frequency and strength mapped to physiological zones) of the “sending” brain's emanated signals, the fuzzy logic feedback learning based, identification of the “receiving” brain's internal physiological locations, modulated receiving frequency and strength)). It needs be noted that each of the two brains would alternately be the sending and receiving brain, for purposes of familiarization of the data contents, initially towards the objective of creating the integrated brain system with one center of consciousness, then for Mary-I to transfer onto the other brain towards creating Mary-II-B (and both brains will have already previously agreed to this transfer so that transfer would typically not need to be consequent to a new decision after the creation of the integrated brain). For speedier and greater practical effectiveness, brain-physiologically-functionally meaningful ranges of signals detected in the “sending” brain should be modulated at meaningfully comparable strengths, frequencies and durations at the “receiving” brain at the latter's corresponding physiologically-functional reasonably equivalent micro locations, over electromagnetic waves at least at the “receiving” brain and preferably also detected as electromagnetic waves in the “sending” brain (since electromagnetic waves would enable optimizing (the process of) interbrain signal interactivity over a meaningfully variable range of modulated strengths, frequencies and relevant physiological locations at the “receiving” brain in the context of the counterparts of the “sending” brain). Extreme accuracy of physiological locations will not be important (only) at the commencement of communications, just reasonable micro-focus on output/input brain components, as signal attributes can subsequently be tuned from the feedback of the participating individuals as well as automatically directly from the neuronal responses at the receiving brain, and furthermore the brains would learn to recognize the signals and optimally adjust for the best sensing and interpretation of the signals. Operations research, numerical analysis and stochastic techniques, coupled with neural networks would optionally enable identifying the participant set specific fine tuned communication signal parameters, including, and of course not limited to, the component micro 3D coordinates internal to the corresponding brains. The logistical flexibility of using wireless electromagnetic waves at both the sending and receiving brains as elaborated in this section would enable the aforementioned computation premised search-and-find-for-optimality micro focus to be extended to if necessary the individual neurons, even to the level of the individual dendrite and axon (and allied) connections per neuron, but in the most part that would not be necessary because (a) there is sufficient neuronal redundancy of distribution of the same bit of information and (b) the equivalent location neuron counts across different brains would be substantially different so a super-micro-mapping would be inapplicable. The micro-focus process can even be expanded upon at the end (i.e. after appropriate focus maximization with electromagnetic devices) using atomic- and/or molecular particle based sensing devices, but that too would not be necessary in the most part. Search and find would essentially need to be targeted on the maximum feedback from participants and more importantly on the maximum automated neural responses at the receiving brain corresponding to the signal received from the output counterpart, at the sending brain, upon commencing the process at the approximate focused locations of known input/output counterparts, and honing in with the stated mathematically optimized techniques premised on the nature of non-optimized (only to reduce the search time—search without mathematical optimization would also work but would take longer, potentially infinitely longer in a few cases) searches until the maximal feedback from the participants and more importantly the maximal neuronal responses at the receiving brain is obtained that would indicate a successful link for that specific micro-component input output pair at the corresponding brains. Again, the data transfer needs preferably be in both directions (either simultaneously and/or alternately) so the sending brain receives real time feedback from the receiving brain and adjust suitably. Furthermore, and separately, and as indicated elsewhere in this Patent Application, from a brain learning and managing perspective, the data transfer needs preferably be in both directions (again either simultaneously and/or alternately).

As also indicated elsewhere in this Patent Application, as part of the connection process, the two linked brains are to be alternately (and sometimes jointly) rendered asleep or otherwise non-willed to allow each brain to learn to be aware of and function with the memory banks and consciousness components of the other brain.

While electrodes could be useful for physiological zone counterpart mapping at the brains, they aren't necessary and are not recommended for this invention, because of the need to participant-specific-variably-micro-“search”-and-“find” optimized input/output brain coordinates, which would be best possible with micro coordinate focused electromagnetic signals—that can (unlike with electrodes) be easily refocused anywhere—of the relevant frequencies, strengths and durations. Hyper fine tuned (per the above techniques) parallel connections of the consciousness- and memory bank relevant corresponding components (supported by the pertinent functional/communications accessories of course) in the two brains would be an additional consideration. Focus should primarily be on the regular communications channels internal to the brain (i.e. generally excluding the external input path components, for speed) so that key input information is laid down in a way that the receiving brain deems optimal, rather than using attempted “forced” communications via channels not ordinarily used by the human brain which could result in the information not being stored properly.

To the extent necessary, a chemical connection with neurotransmitters, as well as a direct, neuronal connection could and should also optionally be considered, as a catalyst to manifesting and optimizing inter-brain communication.

Although memory (and hence also the additional allied) data transfer is from a memory subcomponent buildup (i.e. bottom up) perspective (whereby the memory subcomponents are acquired with equal or more priority as/than are the higher memories that those subcomponents make), alternative consideration would be to allow the sending brain to consciously (and separately with altered, typically diminished, and at times raised levels of consciousness) “load” (i.e. become aware of to a degree of clarity that may be perceived by the (other) brain connected to this brain) relevant memories from its data bank that that brain deems relevant to the transfer and then enable only those memories to be transferred to the other brain. The advantage of this process would be that information would be relatively easily recognized by the receiving brain. The shortcoming would be that the process would be slow, often not include much of the memory (because of the sending brain's need to selectively load a memory first), and needs to be appropriately set up to not compromise or otherwise hinder creating the integrated, augmented, logical center of consciousness—i.e. the center of consciousness of the receiving brain must be suitably tuned to not simply consider the perceived (by it—the receiving brain) memory uploaded by (and at) the sending brain akin to just seeing new images and store that information (in it—the receiving brain) without, attempting to integrate with the sending brain.

Again, the key here for feasibility would be not to reinvent the wheel, but provide a supporting environment (that can be easily optimized per feedback from the two awake brains) conducive to enabling the brains' neurons to do what is natural to them—optimally integrate and communicate.

Any interbrain communication already patented, or otherwise not patentable, today is excluded from the scope of originality of this section, but such technologies are firmly denied the right to be applied to any aspect described as being within the scope of this Patent Application (Ref. “Fair scope of invention” and “Specific steps in invention”) without permission from this inventor.

Albeit already adequately clear above in this section appropriate additional technical elaborations on the above connection technologies may be provided with the non-Patent Application.

### SPECIAL ADVANTAGES OF INVENTION

This approach is in principle a guarantee to perpetual continuity of life, provided of course that a transfer is done prior to Mary-I's death. Even terminal physical ailments in Mary-I would not be impediments to that continuity, as long as Mary-I's brain is still reasonably functional. In fact even mental illness too would be naturally corrected as long as the transfer is done to a brain without mental illness.

This invention pertains not to the speed of the process or emphasize on specific transfer mechanisms, but is the process itself. With time, as the transfer techniques speed up, the procedure can be applied for emergency circumstances when the Mary-I assesses a high likelihood of imminent death, for example when in an airplane that is about to crash, etc. In such scenarios, Mary-I would immediately wirelessly functionally integrate with the other brain located elsewhere outside the risk zone, rendering her brain and the new brain a single functional entity and “transfer” over.

The invention can subsequently be easily extended to primates or even animals of lower cognitive abilities.

### What this Patent Application Does not Cover

This Patent Application does not cover the supporting technologies by themselves (that, when applied in the context of this Patent Application, would enable the brains to communicate) other than the supporting technologies that have been described in this Patent Application as being original—in particular in the section “Technology enabled interbrain communication”. Unless the applicant subsequently acquires patents on those technologies themselves, other inventors are free to develop and patent those supporting technologies to the extent permissible by law and use them for purposes other than that described in this Patent Application. However, all applications of current and/or future technology/-ies towards the following are obviously included within the lawful scope of this Patent Application: the scope of manifestation of perpetual persistence of human life as defined in the subsection “Fair scope of invention”. Although as earlier affirmed, the entirety of this Patent Application contains the applicant's own views (except of course the already medically affirmed components of consciousness of the brain comprising of the centromedian nucleus and the allied, and when implicitly or explicitly declared otherwise in this Patent Application) this Patent-Application also does not cover any aspect of the justification/validation/proof of this invention that might already have been previously patented by others in any other context, or is not patentable.

See “Fair scope of invention” for details on the scope of inclusion of just Step A (“Fair scope of invention”) or Step 1 (“Specific steps in invention”) by themselves in this Patent Application.

The coverage scope as detailed in “Fair scope of invention” will not be superseded by this section or any other section of this Patent Application.

Proof of enablement in principle of direct communication between human brains (termed “interbrain communication (IBC)”) towards unlimited extension to human lifespan

This section (extending for the remainder of the “DESCRIPTION OF INVENTION” of this patent application) (hereinafter referred to as “this section” or equivalent) presents the technical enablement of directly linking two human brains to establish viable direct communication between them, termed “interbrain communication (IBC)” by this inventor (and applicant), in the context of the original patent application filed by the same inventor (and applicant) on unlimited extension to human lifespan. The contents of this section elaborate on aspects of that original invention (of title “Medically perpetual persistence of essential human life”), that (elaborations and aspects) are either expressly or deemed reasonably implicitly included in the original patent application, hence this section's contents are deemed appropriately protected by patent law towards that same prophetic invention as well as towards the same original patent application and therefore also towards this patent application with the original priority. The IBC mechanism and by and large the overall invention also extend to primates and other animals.

### Keywords

IBC (interbrain communication), dendritic spines, corpus callosum, anterior commissure, event related potential (ERP), artificial synapses, stem cells, telencephalon, diencephalon, thalamus (in particular its centromedian nucleus), hippocampus, amygdala. Independent Component Analysis (ICA), (Multi-way/Kernel) Principal Component Analysis (PCA), Parallel Factor Analysis (PARFAC), signal decomposition, theta (4-7 Hz) and alpha (8-12 Hz) frequencies, neuron-to-glial-cell communication, protocadherin, CREB, NR3A NMDAR, entorhinal cortex, basal ganglia, precuneus, MEF2C, channelrhodopsins, synapse strengthening proteins, etc.

### BACKGROUND

The original patent application for the invention that this section primarily aims at explaining the reasoned feasibility of the IBC portion of is PCT International Application No. PCT/IB2009/052355 filed with RO/IB on 3 Jun. 2009 (hereinafter referred to as “original patent application” or equivalent for the remainder of the “DESCRIPTION OF INVENTION” of this patent application (i.e. this section)) that incorporates by reference the entirety of USPTO Provisional Patent Application No. 61058253 filed on 3 Jun. 2008.

Not only would the invention enable human lifespan to be extended without limit for a healthy individual—it would in most cases even also enable the same for someone who is ordinarily deemed terminally ill or someone who has severe physical disability.

### OBJECTIVE

This section highlights, and explains the reasoned feasibility of, some of the deliberated core technology approaches towards practically achieving IBC towards the stated perpetual persistence of human life objectives of the cited invention. For the reader's maximum comprehension of the most significant core aspects of the aforesaid IBC technologies, technical terms and non-essential technical details have been largely excluded from this section.

### Governing Premises of Invention

As indicated in the original patent application, the invention's key objective is for perpetual persistence of human life to happen neither by attempting in futile to perpetually extend the life of the human body (which will never succeed) nor by attempting to merely create a biological “clone” of the entire human body or of the memory contents, as even theoretically exact biological clones would still be mere copies of, and not, the original and therefore (clones) would have the critical shortcoming of NOT being able to allow the original human deem the clone as herself/himself (i.e. the biological clone will NOT have the same, but will instead have a mere copy of, the sense of “I” that the original human will have) notwithstanding that that original human and others around that clone would correctly identify the clone as an accurate copy of that individual; therefore the original human would remain as mortal as ever with the aforementioned futile attempts at human lifespan extension regardless of whether or not her/his biological clone is created. Instead the invention aims to enable perpetual persistence of human life by transferring the brain's “self identity” (i.e. “consciousness” of the self, inclusive of consciousness-, subconscious- and unconscious awareness components) and allied essential and non-essential memory data (with that “self identity” and memory data, et al together constituting the same, not merely a copy of the, sense of “I”) to another brain, and in time from that second brain to yet another brain and so on in perpetuity. As indicated above, the phrase “consciousness” of the self purports to include not just conscious awareness of the self, but self awareness at the sub- and unconscious levels as well. One of the fundamental inferences, and therefore one among the core proofs/justifications/validations and governing premises and key feasibility controls, of/in the invention, is that any individual's specific self identity—therefore the specific “sense of I” originally belonging to that individual—could never be merely copied to be “active” while the original self identity is already still active, but can in principle be only transferred to (i.e. be rendered “active” in) only one logical center of consciousness (e.g. in one brain (or equivalent)). Another of the fundamental inferences of/in, and one of the key feasibility controls and governing premises of, the invention is that such “self identity” data is never tied to any specific brain (or other) hardware—in other words any brain is no more than a provisional holding/containing environment for, and is not itself (and is not uniquely necessary to hold/contain), the self identity of the individual who has that brain.

Another fundamental governing premise and a key feasibility control is that the entire original contents of any human brain are not necessary to uniquely and adequately define (i.e. uniquely identify the external manifestations of the functioning of) that brain—and therefore on extension (uniquely identify) the human who has that brain. Therefore, the entirety of a brain's conscious-, subconscious- and unconscious memory data would not need to be transferred to the brain of another human for adequately “transferring” the self identify of the particular human who has/had that (first) brain. Explained simply, just as when a human loses a limb (or surgically adds new limb(s), or grows massive quantities of new cells across the body as anyway naturally happens to extremely significant extents to every healthy human across the lifetime of that human) the remainder (or the partly or fully upgraded augmented rendition) of the human still remains uniquely and reasonably adequately identifiable (both to that specific individual's own self and to others) as being no other than that same original individual and appropriately functioning with/or without added prosthetics, and similarly just as when portions of a human's brain are lost to TBI (traumatic brain injury) or to surgery (or massive quantities of new brain cells are added to the brain as naturally happens to very significant extents to every healthy human brain across the lifetime of that brain) the remainder (or the updated partly or fully augmented rendition) of that human brain can still remain uniquely and reasonably adequately identifiable (both to that specific individual's own self and to others) as belonging to (i.e. manifesting) that same original individual and appropriately functioning without the individual's losing self identity (except in rare situations when conscious self identity is lost when the brain is too damaged, yet even then aspects of the sub-/unconscious identity and other functions of that brain often still remain sub-/unconsciously identifiable to the self, and consciously to others) with or without corrective/other alterations to the brain, the quality of the success of self identity transfer, hence the degree of effective perpetual persistence of life—i.e. the quality of effective biological immortality—of that specific human, would essentially be a function of the extent of the memories, primarily those related to conscious cognition and secondarily also including those that are not (namely sub- and unconscious memory data), defining that human that are transferred.

This premise is further justified by the fact that albeit every human's conscious and subconscious (and to relevant extents unconscious) memory contents are substantially different from the corresponding renditions 1 minute or 30 days or 1 year or 10-/20-/50 years ago by way of considerable alterations—primarily additive, secondarily modifying and/or subtractive—to the conscious-, subconscious- and unconscious memory content, each of the varying renditions of that same human's overall memory content essentially fully retains her/his identifying (both to that specific individual's own self and to others) uniqueness and to appropriate extents her/his adequacy of functioning.

Restating the above further, even when a huge volume of new memory content is added, and in most cases when much of the memory content is modified or lost, the person is able to consciously/subconsciously/unconsciously recognize herself/himself as being the same original person, as too do others around that person.

While the conscious aspects of memory (i.e. declarative memory (comprising of episodic and semantic memory)) would constitute the most prominent parameters uniquely defining any particular human's self identity, the sub- and unconscious memory components, learnt (i.e. procedural memory) and otherwise (i.e. genetically pre-existing sub-/unconscious “memories”, i.e. genetically pre-programmed neural attributes), not entirely or even at all related to conscious behavior, also contribute to uniquely defining that human's self identity—for example by way or the manner the human walks, laughs or otherwise interacts with society, other unique behavior or mannerisms of that individual, and even less significant, involuntary aspects as how that particular human breathes, etc. In other words, the identifying signature; of not just the brain's conscious and sub-/unconscious “memories” but even of the peripheral nervous system, including both its somatic and autonomic components—the latter's both parasympathetic and sympathetic categories—contribute to that human's self identity, although to a considerably lesser extent than the conscious memories. Therefore while IBC on conscious memory components would be the overwhelming core aspect of this invention, IBC on sub-/unconscious memories would also have a role, and to a very minor extent even IBC relevant to the peripheral nervous system.

It is important to note that the objectives of the IBC would include both to “transfer” (as in copy) memory data as well as to “transfer” (as in move) self identity. See the four steps listed in the original patent application (Ref. section “Specific steps in invention” on pages 10-16 of that original patent application) body for details.

For purposes of this invention, the areas of the brain that would be primarily, but not only, focused on towards enabling direct IBC with would be the memory and cognition sites in the telencephalon and diencephalon of the prosencephalon.

### Feasibility of Invention

The original patent application cited in this section lists a total of five (5) key bases of feasibility of the invention (Ref. section “Key feasibility controls” on pages 8-10 of that, patent application). Those details are not comprehensively reiterated in this section; some of them have been (e.g. in section “Governing premises of invention” of this section, and in this section).

The feasibility of IBC itself has been covered at length in this section.

Albeit, owing to the complexity of the core innovations constituting the invention, the latter's proof of feasibility cannot be comprehensively expressed in simple terms (the entirety, not mere portions, of the original patent application requires the reader's meticulous, competent comprehension towards her/his comprehensive technical understanding of why and how the invention is feasible inclusive, and because of, its innovations), surmised below is the proof of the invention's feasibility in stripped down, simple, non-comprehensive terms constituting another legal proof of the invention's feasibility without detailing on the innovation aspects of the invention (other than naturally reflecting the cogently compelling innovation quantum extant in the following core approach itself towards enabling perpetual persistence of human life):

Brain neurons are already naturally highly versatile to essentially unlimited extents in terms of their ability to directly link up to (using newly generated intermediate neuronal pathways and/or existing neuronal pathways), and therefore communicate with, other already existing appropriate neurons in that brain, so there's no reason why, with the right, “reasonable” technology “aid” (i.e. with IBC), the neurons of one brain wouldn't naturally be able to directly link up and communicate with appropriate neurons of another brain. Such forms of neuronal communication are also already noted for example with transplanted organs/limbs.

Every healthy human brain naturally grows, at the appropriate time points in its lifetime (i.e. ranging from a very young age from before birth up to an elderly age), massive quantities of new neurons and integrate such neurons into that (regularly upgrading) brain causing almost every anatomical area of the brain to grow physically larger than it was before (as happens when the brain is still young) or (at least) be modified even when not physically larger (as happens both in young and older ages), allowing the original memory content and new memory content and (other) brain functions to continue to be appropriately integrated across that being-continually-physically-upgraded brain (with all earlier and newer memory data accessible as usual), and the brain still continues to have a single “logical” (as opposed to physical, as various physical locations in that brain are necessarily involved) center of consciousness—i.e. a well integrated single self identity continues to manifest (where the sub-components of self identity from the integrated memory data are all duly integrated, yet are still appropriately duly separately present for easy natural separate access willfully or otherwise). It is also scientifically well recognized that the extent and efficiency of the process of the overall memory encoding and retrieval and other capabilities of any brain can generally be substantially increased by deliberate natural effort/exercises in the brain. Therefore there's no reason why, with IBC and some deliberate natural effort/exercises in two brains, the same cannot happen with those two different brains—forming one integrated, single, “logical” brain (as opposed to one physical brain, since two physical brains are involved) with integrated memory data (i.e. combined memory data from the two brains, yet all memory data individually accessible post integration) and an integrated, single logical center of consciousness (and a well integrated single self identity formed of the two separate self identities (per the memory data) in the original two separate brains, yet those two separate component self-identities still appropriately duly separately present for easy natural separate access willfully or otherwise).

Because of the typically considerable multi-redundancy of both data storage and functionality within any healthy human brain, including the ability of the human brain to adjust to new data and develop functions anew when necessary, the healthy human brain is reasonably capable of appropriately adequately functioning even when portions of any of its functional/anatomical areas are naturally discarded or even suitably removed surgically. Therefore there's no reason why the integrated, single, “logical” brain of the above paragraph cannot have the first physical brain physically removed from it (by terminating the IBC following the conclusion of the action surmised in the above paragraph) with no loss to the integrated memory data and the integrated single self identity in the second brain. As indicated in the above paragraph the integrated single self identity and integrated memory data will already include memory and self identity from the first brain (integrated with, yet accessible separate from, the second brain's original memory and self identity), per this invention's objective.

The memory data contents of any healthy brain are being constantly increased (and to a certain extent modified or lost) across the lifetime of the brain; yet there is no loss to self identity (even if normal multiple (i.e. varied flavors of) “personalities” are present, which is a very common normal phenomenon for most humans). Therefore there's no reason why the integrated memory data in the second brain post-IBC-termination of the above paragraph would in any way compromise the integrated single self identity (duly composed of the two separate original self identities, each individually accessible at the integrated will of that integrated single self identity). As indicated above in the earlier two paragraphs the integrated single self identity in the second brain will have self identity from the first brain along with the self identity that the second brain originally had.

### Feasibility of Interbrain Communication (IBC)

The feasibility of IBC is analyzed in this section under two sub-sections, one on the specific areas of the brain to focus IBC on, and the other on the specific technology particulars to use for such IBC. Some readers might prefer to review the IBC technology subsection before reviewing the subsection on brain areas.

### Specific Areas of the Brain to Focus IBC On

The essential approach in the invention is a 1:1 component-mapped IBC simultaneously for all IBC-relevant components of count n in the two brains, where n<N, N being the count of all (i.e. inclusive of non-IBC-relevant) corresponding components in each of two brains. Additional considerations are to simultaneously, spanning all n components, have grouped m:m, m:M or M:m IBC, where m<M<n<N with the groups being functionally relevant, to allow greater efficiency yet ensuring that IBC data integrity would remain as good as in the 1:1 architecture because any irrelevant input signal (from the other brain) received over IBC at any member subcomponent of any group of m or M components would be automatically recognized and discarded by the brain.

The brain areas listed in this subsection include a majority, but not all, of the core areas to focus IBC on. Certain areas of the brain are mentioned more than once appropriate to their functional relevance to IBC. The areas are not listed in any particular sequence, other than by and large listing the IBC relevant components in the prosencephalon (constituted by the telencephalon and diencephalon, not necessarily covered in that order), mesencephalon and rhombencephalon in that order.

Before getting into IBC details per the above ordering it may be noted that the prefrontal cortex, amygdala, hippocampus, precuneus and other areas of the prosencephalon play significant roles in declarative memory; hence those regions would be highly relevant to IBC. The basal ganglia and the cerebellum have roles in procedural memory and hence are IBC relevant, albeit not to the extent of the brain components associated with declarative memory.

The neocortex (neopallium), hippocampus and amygdala of the telencephalon and the thalamus, hypothalamus, subthalamus and epithalamus of the diencephalon are among brain areas to be importantly considered for IBC. The neopallium subcomponents frontal cortex (specifically including and necessarily not limited to its prefrontal cortex), parietal cortex, anterior cingulate and parts of another component of the telencephalon—the basal ganglia—would be of high IBC significance.

Sections of the limbic system, namely the thalamus (including but not limited to the centromedian nucleus), amygdala, hippocampus, entorhinal cortex, limbic cortex, anterior thalamic nuclei, mamillary body, fornicate gyrus (inclusive of, but not limited to, its cingulate gyrus and the inferior parahippocampal gyrus), fornix, dentate gyrus, orbitofrontal cortex, and its other constituents would be among IBC priorities. It might be noted that there is not a universally accepted defined set of constituents of the limbic system.

The amygdala, for its role in modulating memory particularly but not limited to in the context of emotions, would have its IBC priority in this order: cortical nucleus, central nucleus, medial nucleus, basolateral nucleus.

Dentate gyrae, deemed part of the hippocampal formation, would be IBC relevant because neurogenesis occurs here allowing the formation of new memories, for example for the possible distinction between multiple memories of similar incidents.

The thalamus forms thalamo-cortico-thalamic circuits that are deemed critical to consciousness, and its role towards appropriate inter-brain memory data interchange with IBC towards individuality manifestation for perpetual persistence of human life would be vital. Among the most pertinent areas of the thalamus would be the centromedian nucleus (part of the intralaminar nucleus), also known as the centrum medianum (Cm-Pf/CM). The CM is known to have both excitatory (e.g. over acetylcholine) and inhibitory (e.g. over Gamma-Aminobutyric acid (GABA)) effect on the, corpus striatum. The medial dorsal nucleus is part of the thalamus and is connected to the limbic system and to the prefrontal cortex, and it has key roles in memory, abstract cognition, planning et al and will be part of IBC considerations.

The hippocampus is among the most important components relevant to memory formation, and would be a key IBC factor. It is believed that with the passage of time long term memories are formed outside the hippocampus. The key circuitry for the hippocampus involves receiving input from the relatively superficial layers (EC2, EC3) of the entorhinal cortex (EC) and sending output to the deeper layers of the EC. The cornus ammonis 3 (CA3) area of the hippocampus receives input from EC2 as well as from the dentate gyrus (DG, deemed part of the hippocampal formation as indicated previously in this section), while the cornus ammonis 1 (CA1) receives input from EC3 as well as from cornus ammonis 3 (CA3) (through the fibers of the “Schaffer collaterals”). The perforant pathway connects the entorhinal cortex to all the CA components of the hippocampus, as well as to both the subiculum and the dentate gyrus areas of the hippocampus. CA1 also receives input from the nucleus reuniens of the thalamus. Output from the CA1 goes to the deeper layers of the EC, either directly or via the subiculum (which is part of the hippocampus). This is represented in the circuit diagram in FIG. 3.

It is deemed that areas of the neopallium (a.k.a. neocortex/cerebral cortex/isocortex) and the hippocampus synchronize information and functioning in the theta frequency for various cognitive operations, including but not limited to for working memory activities.

Therefore the theta frequency would be an IBC synchronization basis for those components across the two brains.

As has been mentioned in greater detail in the IBC technology section of this section, this invention deems it meaningful to tap directly into the signals at inter-hemispheric neuronal pathways for IBC towards easier access into the respective hemispheres. Important among such IBC relevant inter-hemispheric neuronal connections are the corpus callosum, the anterior commissure and the hippocampal commissure (also known as the commissure of fornix).

The parahippocampal gyrus surrounds the hippocampus and is one among gyri that are part of the temporal lobe of the cerebral cortex (neopallium), and it is relevant to encoding and retrieving memory, hence pertinent to IBC. The parahippocampal cortex is a specific area combining the (medial) fusiform face area (FFA) and part of the (posterior) parahippocampal gyrus. The FFA complements the function of the PPA (which is part of the parahippocampal gyrus) in that the former is functional in the context of recognizing faces, while the latter is functional for scene recognition.

As with the entire above portion of this subsection, unless otherwise mentioned, all the components listed below in the remainder of this subsection would play a role in IBC.

Included in the (anterior) parahippocampal gyrus are both the entorhinal and perihinal cortices, both of which merit IBC. Forming the primary input to the hippocampus the entorhinal cortex (EC) (Brodmann areas 28 and 34) preprocesses the signals input to the hippocampus (for familiarity/recognition). Data connection with the EC is with the hippocampus, the prefrontal cortex, the perihineal cortex, etc.

The fornix of brain is a route for signals to the septal nuclei and mammillary bodies from the hippocampus, and deemed to be relevant to memory, hence is compatible to IBC considerations.

The mammillary bodies are material to IBC in that, among other functions, they route signals to the thalamus (via the mammillo-thalamic tract) from the hippocampus and amygdala, and are believed to have a role in recognition memory and smell memory.

The Papez circuit (FIG. 4)—a key circuit of the limbic system deemed significant to long term declarative memory encoding and (deemed by some) to control emotion—surmises some of the overall key connections of relevance to encoding memory, and hence its components are IBC pertinent.

The orbitofrontal cortex consists of Brodmann areas 11, 47 and 10 in humans, and is involved in decision making. It merits IBC considerations.

The medial temporal lobe (MTL) overall is thought to be associated with declarative memory, awareness and learning and would be relevant to IBC. Old memories are distinguished from new by the MTL regardless of consciousness state. Along the same lines, learning is facilitated by the MTL regardless of conscious awareness.

The basal ganglia is (are) highly germane to IBC, including but not limited to in the context of procedural memory. It is part of the telencephalon and is primarily comprised of the subthalamic nucleus, paleostriatum (a.k.a. globus pallidum), striatum (a.k.a. striate nucleus) and the substantia nigra. The caudate nucleus, which is part of the striatum, has learning and memory function. The putamen, which too is part of the striatum, has a role in various forms of learning, in particular but not limited to category learning, and although the exact functional specialization of the putamen is unknown the fact that it is connected to key brain areas and therefore and otherwise is deemed important to learning renders it notably IBC relevant to the project. The subthalamic nuclei, which are part of the basal ganglia, will also be specifically considered for IBC.

The primary motor cortex, which is part of the frontal lobe of the cerebral cortex, works along with the pre-motor cortex (Brodmann area 6) towards planning and making decisions. The supplementary motor cortex is relevant to memory and learning. The primary-, pre- and supplementary motor cortices will all be part of the IBC scope because the motor skills that they relate to would constitute part of the self identity signature.

The superior frontal gyrus—a major component of the cerebral cortex and an important IBC focus—has a key role in self awareness. Importantly, the inferior frontal gyrus is associated with verbal skills (Brodmann areas 44 and 45 constitute Broca's area for syntactic/semantic and phonological processing) and on selective decision making, hence it has IBC relevance vis-à-vis speech and decision signatures. While the superior- and inferior frontal gyri would be relatively important IBC candidates of the project, the middle frontal gyrus would be less so. The fusiform gyrus, by use of its FFA (Fusiform Face Area), would be a notable IBC candidate, for the reason of its playing an important role in recognizing faces and other objects.

Several among Brodmann areas associated with the temporal lobe would be appropriately assessed for IBC considerations. For example, BA20 (deemed to be associated with recognition memory), BA34 (also part of the entorhinal area), etc are relevant in that context.

The precuneus of the parietal lobe would have IBC relevance on account of its role in episodic memory, consciousness and self-awareness.

Among the other areas of the parietal lobe, the primary somatosensory cortex (S1, comprised of BA3-1) and the posterior parietal cortex would be low-to-moderately meaningful candidates for IBC on account of the individual identifying signatures they enable in the context of processing sensory and allied stimuli. The former is significant vis-à-vis sensing touch and other stimuli, and the latter enables planning by appropriately integrating motor and sensory functions. The postcentral gyms of the parietal lobe has functions that have some similarity to those of the posterior parietal gyrus, and would be a moderate IBC candidate for the project.

The insular cortex has several significant roles, including awareness of the body's internals, awareness of the self and control of emotions, control of motor functions, etc. and it would be an IBC candidate.

The cingulate cortex would be of IBC relevance, and appropriate of its segments—subgenual area 25 (BA25) (plays a role in the hippocampus pertinent to memory), anterior cingulate (essential to working memory), retrosplenial cortex (RSC) (relevant to memory)—would be given due IBC consideration. Both the parahippocampal place area (PPA) and the retrosplenial cortex are critical to spatial navigation, with the former focusing more on spatially interpreting the immediate local visual data while the latter on placing that data in the overall spatial context. The dorsal anterior cingulate cortex (ACC) effects cognition, while the ventral ACC is pertinent to emotion—so while both would be IBC candidates the former would be relatively more significant.

Brodmann areas 41, 42 and 22p (essentially comprising the primary auditory cortex and Wernicke's area) of the superior temporal gyrus would be IBC considered. The inferior- and middle temporal gyri would be relevant to some extent in as much as their visual processing (i.e. visual memory) signatures.

The mesocortical-, mesolimbic- and nigrostriatal dopamine pathways would be IBC contemplated.

The Nucleus Basalis of Meynert (NBM) of the basal forebrain is deemed relevant to attention, and hence would be material to IBC.

The temporal neocortex would be looked at for IBC, being where long term memory of the initial temporary memory is formed at the hippocampus.

The mesencephalon (mid brain)'s tectum has functions related to auditory (inferior colliculus of the tectum) and visual information processing (superior colliculus of the tectum), allowing it to carry information processing signatures (as different individuals would uniquely differently process the same visual/auditory information) that would uniquely identify a specific individual, rendering the mesencephalon moderately relevant to IBC. Much of the mesencephalon other than the tectum is the cerebral peduncle, which is often defined as being inclusive of the midbrain pretectum, tegmentum, and the crus cerebri.

The metencephalon and myelencephalon—of the rhombencephalon—would not be a priority for IBC but they would not be entirely excluded, because the sub- and un-conscious memory signatures they carry are unique to identifying specific humans. Of these two, the pons and cerebellum in the metencephalon have reasonable IBC relevance. Listed below are some of the functional areas where such signatures exist. The pons routes certain sensory data between the cerebellum and the cerebrum, and the pontine tegmentum of the pons has a role in REM sleep (and therefore in consciousness). The density of P-(ponto-geniculate-occipital)-waves in the pontine tegmentum increases following memory training activity, hence the inference that the P-waves are, and hence the pons is, relevant to learning. The parabrachial nucleus (PbN) has a role in taste sensation, including for signals received from the nucleus of the solitary tract (NTS). The pons also has a role in the synchrony between respiration and cardiac activity characteristics, thereby indirectly constituting one among brain centers associated with involuntary identifying signatures pertinent to IBC.

The cerebellum carries memory signatures by virtue of its important role in procedural memory, hence is relevant to IBC, in that it, among others, contributes to motor control with perception of sensory data, by enabling a coordinated integration of data exchange between the motor cortex of the cerebrum and spinocerebellar tract. The cerebellum in particular the neocerebellum—also has cognitive functions including but not limited to attention, of some IBC pertinence.

### IBC Technology

i. Core Approach

For purposes of IBC quality and efficiency, every effort would be taken to refrain from reinventing “the wheel” from ground up, but rather by letting the brains maximally naturally communicate and acquire data content by themselves from each other in a signaling technology environment.

IBC would be an optimized functional and physical (i.e. anatomical) integrative process. In other words, IBC will be undertaken based primarily on anatomically equivalent locations of the two brains, the selection of which (locations) would be suitably optimized or modified based on functional equivalence, as precise functional equivalence might not always be identifiable by only anatomical or only functional equivalence alone. Towards that integrative process, to identify the approximate functionally equivalent locations, the two brains would be subject to a very wide range of categories of external stimuli (e.g. static and video images, language- and non-language audio and video (examples of language related video would be written text, video of people speaking, visually symbolic language), simple through complex logic processing scenarios that would evoke both regular-data-memory and logical-ability-(i.e. unique logic signature)-memory, olfactory input, tactile input (including but not limited to when combined with language), other memory evoking and allied memory relevant external input, etc) and internal stimuli (i.e. primarily electrical and electromagnetic (and optionally biochemical) stimuli applied internally directly into locations of anatomical relevance (including but not limited to information already publicly known on elaborate input/output sites of various neuronal circuits in the human brain) coupled with the aforementioned earlier-stage location conclusions consequent to the external stimuli) and the corresponding integrated locations and the respective brainwave frequencies would be noted. Those integrated locations in the two brains would then be identified with increasing accuracy in several stages by iterating the above process multiple times. Then IBC would be established between the two brains at those locations at (or otherwise compatible/synchronized to) the appropriate wave frequencies (including but not limited to in synchronized theta (4-7 Hz) and alpha (8-12 Hz) frequency ranges, given that those frequencies are deemed highly relevant to memory encoding and retrieval), strengths and durations—including but not limited to as a variable sequence of “signal bursts” of electromagnetic and/or electrical signals, while noting that IBC would (also) be appropriately directed to the respective input/output neural circuit areas—i.e. the output of a component of one brain fed to the input of the equivalent component in the other brain (instead of and/or in parallel addition to naturally feeding that output to the equivalent component in the same brain itself as well) and vice versa. The beta (12-30 Hz) and gamma (>30 Hz) frequencies would also be used for IBC, both when those are the base frequencies of the corresponding brainwaves at those locations as well as otherwise for augmenting IBC efficiency and quality. The categories of synchronizing the IBC signal with the brains that would be considered would be in-phase synchronization where there would he no phase shift and the frequencies match, out-of-phase synchronization where there would be a phase shift that could be greater than zero and less than half of the phase, anti-phase synchronization where the frequencies would be the same but there would be a half-phase shift, only-frequency-synchronization where the phases could be anything but the frequencies match, partial synchronization where in a large group of neurons some (typically the majority) would operate in synchrony but the rest wouldn't and the whole group would still maintain the overall synchrony, coincidental synchronization where there would be no uniform frequency or phase but nevertheless the probability would remain high that specific events (e.g. overall group of maximum or minimum spikes, etc) of the operations would match the corresponding events of the other, virtual modulated synchronization where the frequencies would be in integral multiples and therefore in a virtual phase. The reason for the need to consider all the above frequencies and phases is that two brains will not necessarily operate in the exact same frequencies and phases even at the earlier mentioned considerably precisely identified functionally equivalent sites: hence optimal frequency-phase-intensity-duration combination(s) corresponding to various functionally equivalent sites across the two brains would need to be identified specific to those two brains.

As mentioned above, external stimulus alone would not be adequate to evoking a sufficiently large domain of memory. The reason for that is that large chunks of memory would not have been originally created based on external stimulus alone, because much of such memory would have been formed consequent to logically variably internally processing (often involving a huge array and layers of internal logical processing and memory accesses) complex-external-input with already existing internal conscious-, subconscious- and unconscious memory data, hence such memory couldn't ordinarily be invoked by external input alone. Another reason for the need for direct internally applied memory retrieval triggers is that even for memories that have been formed primarily (Note: ordinarily it cannot be only) based on external input alone it would not be always practicable to select, all possible relevant combinations of external input as memory retrieval triggers to retrieve those memories, simply because such external input combination possibilities are too huge and too varied for different people. Accordingly, some of the memory would need to be accessed by direct internal stimulus to (i.e. internal signaling at—typically but not always necessarily electrical or electromagnetic) specific functional locations in the brain itself.

The synchrony in the partial and coincidental synchronization categories, among others, would be determined specifics of with appropriate numerical analysis, Fourier transform, and other processing.

Neuronal synchronization between the two brains would be across a massive count of neurons in each at the same time, over artificial chemical and/or electrical “synapses” located between each end of the virtual electromagnetic and/or electrical (and/or chemical) communications (for IBC) channel and the group of neurons at the two brains. All that would essentially be necessary, with relative technical ease, is to electrically detect the real (i.e. neuronal) action potential at the final (group of) neuron(s) in the pre-synaptic end(s) in one brain at the point it is connected to one (outbound) end of the IBC channel and transmit that electrical voltage—not at all necessarily at the neuron-typical +70 mV but at an appropriate (other) voltage—signal to the artificial synapse(s) at the other (inbound) end of the IBC channel connecting the other brain, which would result in the artificial synapse(s) at that inbound end of the IBC channel releasing the appropriate neurotransmitters (for an artificial chemical synapse) causing the triggering of, or simply electrically (for an artificial electrical synapse) directly triggering, an action potential in the first (group of) neuron(s) connected to the IBC channel at that receiving end, which should result in the natural chemical and/or electrical synaptic communication down the IBC link from one brain to the other, and back in a similar process.

These artificial synapses would be created to function at the appropriate oscillation frequencies pertinent to the categories of the pre- and post-synaptic neurons in the IBC link. Artificial synapses would be complimented by appropriate natural synapses that would (naturally) develop during IBC. It is to be noted that in the context of IBC these natural synapses are those that newly develop between neurons at either of the two brains to facilitate IBC per the brains' natural tendency to transfer, receive and assimilate information. While largely, other than the artificial and natural long-/short term synapses, the remainder of the two brains at the two ends would not need to be physiologically modified too much, some IBC effectiveness- and efficiency enhancing techniques, in part listed below in the remainder of this section, would be considered.

The corpus callosum is deemed in this invention an optional and/or complementing location for IBC connections. The reason for that is that because the corpus callosum already appropriately reaches into the depths of the two hemispheres of each brain, accessing those hemispherical depths would be appropriately relatively easier by directly accessing the corpus callosum itself. The specific connection points on the corpus callosum to enable IBC at would be identified in a mechanism using external and internal input to the two brains and determining which neuronal areas of the corpus callosum activate and integrating that information and the corpus callosum's already known anatomical (including circuitry) information with the corresponding activation and anatomical information for the remainder of the brain. That overall mechanism would be similar to that for identifying IBC connection points in the remainder of the brain as described in this section.

The anterior commissure which links the two cerebral hemispheres, albeit to an overall extent considerably lower than that for the corpus callosum, would be another area to focus IBC at, with proceedings as with the corpus callosum. Focus on the anterior commissure would be primarily, but not limited to, in the context of the temporal lobes and the amygdala pair, with those being among the primary brain components relevant to IBC that they connect across the two hemispheres.

In a manner similar to that for the corpus callosum and the anterior commissure, the hippocampal commissure (a.k.a. commissure of fornix) is important in as much as the hippocampus, because it in effect connects the hippocampus across the two hemispheres.

For IBC between equivalent components in the two brains it would not be necessary for those equivalent corresponding components to be linked in entirety or even to a highly major extent. When the entirety of two equivalent components in the two brains are not directly linked, the communication bandwidth available to the aforementioned directly linked subset of components would be used by the entirety of those equivalent components to communicate with, wherein data in the non-linked portions of the equivalent components of the brains would pass through the linked portions. In fact extreme accuracy in identifying IBC-relevant equivalent components/locations in the brains would not be necessary—reasonable accuracy would suffice—the equivalent components of each brain would know to filter in and filter out relevant and irrelevant signals from the corresponding equivalent and non-equivalent sections respectively of the other brain towards IBC. In fact irrelevant signals exchanged between even equivalent components across the brains would be similarly naturally discarded. However, the efficiency and to a certain extent the integrity (i.e. inclusive of quality) of information transferred (i.e. the extent to which relevant data is not excluded from transfer, and corruption is minimized of pre- and/or post-transferred data) over IBC would be increased with greater accuracy in connecting equivalent locations. A communicating “band” of reasonably high bandwidth, typically using an appropriately large count of virtual synapses, between reasonably equivalent brain components should be sufficient for IBC. Data would be exchanged across those bands between the equivalent areas of the two brains, somewhat akin to, but less extensively than, how the corpus callosum assists the two hemispheres of a brain to communicate. Just as any healthy brain continually naturally learns how to optimally use the corpus callosum depending on the content and other circumstances of the inter-hemispheric data transfer, the two brains linked for IBC would also naturally learn—with the appropriate technology assistance in the directions outlined in this section and in the original patent application—how to optimize the use of that IBC channel. In other words, IBC would happen, albeit inefficiently arid inadequately, towards the invention's objective of perpetual persistence of human Life, even with a relatively rudimentary IBC communications channel. Yet the more advanced the IBC mechanism per the directions outlined in this section and in the original patent application, the more efficient and adequate IBC would be towards the invention's objective of perpetual persistence of human life. The novelty/tangibility/etc—hence the patentability—of the invention is not only in the technology of the IBC mechanism towards that objective, but also significantly in the very fact that perpetual persistence of human life can at all be plausibly feasibly attained with IBC regardless of howsoever rudimentary (i.e. nevertheless always constituting the invention's use of the technology “tool” IBC) or sophisticated that IBC mechanism is.

Illustratively expanding with an example on the point of patent law on the logic of patentability of any invention, an ordinary nail in itself is not novel/tangible/etc as an invention—hence not patentable—any longer, but even if that ordinary nail with nothing else could be innovatively used for any purpose that newly achieves an extraordinarily significant, meaningful human objective even if a significant quantity of natural human biology constitutes a major proportion (hypothetically, even if as high as or higher than 99.99% as long as that proportion is less than 100%) of the overall technology process of attaining that objective, the use of that simple nail to at all enable that objective would constitute a patentable invention.

Various categories of event related potential (ERP)—namely potentials measured in the brains consequent to perception, cognition et al (as part of the earlier discussed overall process of external and internal input towards determining equivalent locations in the two brains for IBC)—would be used to identify the IBC relevant functionally equivalent locations in the two brains. Such ERPs would include, but wouldn't be limited to, N100, N400, P300 (P3), SPS (P600), ELAN (early left anterior negativity), late positive potential (LPP), etc. LPP is deemed relevant in the context of managing emotions, and is therefore deemed pertinent in the emotional memory signature transfer context of IBC. ERP P200 (P2) is deemed significant to the process of intent, and therefore and otherwise in memory. N270, N430 are deemed pertinent to conflict identification and processing. P3a and P3b are cognitively important in that while the former is associated with setting attention to something of adequate newness the latter occurs when unlikely events related to the task already being processed occurs.

Since EEGs contain information from several ERPs and other signals that are superimposed, thereby muffling the independent ERPs, it would be necessary to suitably process the EEGs with Independent Component Analysis (ICA), (Multi-way/Kernel) Principal Component Analysis (PCA) (including the Karhunen-Loève theorem rendition). Parallel Factor Analysis (PARFAC) a.k.a. Canonical Decomposition, Tucker Decomposition, Adomain Decomposition Method (ADM) for solving partial and ordinary non-linear differential equations using non-numerical techniques. Domain Decomposition Methods (DDM), Cholesky Decomposition, Largest Lyapunov Exponent (LLE), Hurst Exponent, Higher Order Spectra (HOS), et al.

ii. Optimization

Signal enhancing techniques need be suitably applied to render IBC faster and more adequate. Techniques surmised below would be among those that would be considered towards that objective.

Embryonic and adult stem cells as well as progenitor cells enabling the growth of new neurons would enhance IBC connectivity.

The CREB (cAMP response element binding) protein, deemed relevant in the formation of long term memories, would be assessed for optimized use at both the sending and receiving brains in the project. The memory augmenting effect of pregnenolone sulfate injected into the limbic system would be a factor considered towards expediting IBC. The memory consolidation effect of down-regulating NR3A NMDAR(NMDA (N-Methyl-D-Aspartate) receptor)s would be assessed, in particular, but not limited to, because it is understood that NR3A—a type of NMDAR—causes resistance to the formation of long term memories by way of reducing synaptic plasticity, (reducing) synaptic counts and (reducing) synaptic densities. PolyADP-ribosylation would be looked at in terms of maximizing long term memory formation. The interaction between NMDA receptors and D1 receptors would also be studied in the context of long term memory formation. Also would be assessed the effect (on long term memory) of the induction of AMPA receptors into synapses. Further assessment would be on the relevance (to working memory) of hippocampus dependent, configuration-relational (CR) synchrony in the thereabouts of the theta frequency across the temporal and occipital lobes and non-CR (non-hippocampal-dependent) maintenance across the parietal and frontal lobes, and the effect that alpha oscillations have on LTP. The effect of dopamine on LTP would also be considered. The effect of CAM kinase II on hippocampal LTP would be assessed. It is deemed that theta rhythms and interactions between theta and alpha rhythms are critical to encoding and recalling memory. Therefore substantial focus would be on theta and alpha synchronization for optimized IBC.

The significance of the MEF2C (MADS box transcription enhancer factor 2, polypeptide C a.k.a. Myocyte-specific enhancer factor 2C) protein to learning in the context of IBC would be investigated. In particular, the deemed reduction of memory (and therefore learning) abilities in specific areas of the brain, including but not limited to the hippocampus, (despite a possible consequent) associated increase in the synaptic densities, by under-expressing (i.e. reducing) MEF2C would be looked into.

Dendritic spines are deemed to be extremely significant to memory. In part because they're based on actin, they are highly plastic—i.e. they change their shape and size (and count) quickly, in particular the former (i.e. shape) which can change in seconds. The key types of receptors that dendritic spines express are receptors for glutamate and receptors for BDNF (Brain-derived Neurotrophic Factor). Examples of glutamate receptors are NMDAR, AMPAR, etc. Dendritic spines are deemed to be highly significant to cognition and short term memory as well, not just long term memory. Accordingly, a brain's ability to communicate effectively and fast would be directly dependent on the efficacy of its use of dendritic spines. The efficiency (i.e. speed) and extent of IBC would be significantly a function of the optimal production and use of dendritic spines in both brains (Note: not necessarily only the use of existing dendritic spines, but the use of dendritic spines specifically created to enable IBC be more efficient and effective).

The role of specific neuropeptides towards augmented neuronal interconnectivity would be analyzed, including but not limited to
Galanin: Of the three receptor classes GALR1, GALR2 and GALR3 (all of the G Protein Coupled Receptor (GPCR) class), significant would be GALR3 which is deemed relevant to memory and cognition. Galanin coexists with the neurotransmitter norepinephrine.
Neuropeptide Y: This neuropeptide coexists with the neurotransmitter norepinephrine (noradrenalin), GABA, epinephrine (adrenalin), and it is deemed to play a role in memory.
Substance P: This coexists with the neurotransmitters acetylcholine and serotonin (5-Hydroxytryptamine). It is specifically associated with pain reception. Artificially induced pain, at various locations, to various degrees and for varying durations, would be considered as an IBC enhancing factor for this project.

As indicated earlier in this section, the optimal production and use of dendritic spines would be critical to optimal IBC, so that huge volumes of memory (and related) data are transferred fast and without compromising the integrity of that data. Of high relevance in this context are the spine apparatus (endoplasmic reticulum) found inside some dendritic spines and the synaptopodin protein that constitutes an important part of the spinal apparatus. The factors that affect the morphology/morphogenesis of dendritic spines would be suitably used towards the production and use of dendritic spines for IBC. Such factors include, but aren't limited to, receptors, proteins for scaffolding, cytoplasmic kinases, enzymes, special proteins and chemicals, and electrical, magnetic and other stimulants, et al. For example, in addition to the AMPA receptor, NMDA receptor etc the SIGMA1 receptor (present at the endoplasmic reticulum) that encodes calcium signaling is deemed to be relevant to dendritic spine creation; enzymes, amino acids and tumor suppressor proteins like Scribble modulates the expression of dendritic spines; melatonin affects spine density; the GluN class (including but not necessarily limited to the GluN2B class) of NMDA receptors are deemed to be highly relevant to affecting dendritic spines in the hippocampus and cortex and thereby affect learning—optimally increasing GluN2B for example could augment IBC efficiency; protein kinase (a.k.a. phosphotransferase, which are enzyme(s) that function to transfer phosphate group(s)) that rely on Calmodulin (calcium binding protein) are also deemed important for dendritic spines in the context learning and therefore to IBC; etc. The effect of three of four classes of the oligopeptide angiotensin—namely classes II, III and IV—would be looked into in as much as enhancing adequate growth of dendritic spines and therefore of the efficacy of IBC. The extent to which the ABI family of genes is able to affect actin polymerization and therefore (its receptors) affect dendritic spine formation would be a consideration in the project. Estrogen would be looked into in as much as its ability to optimize the morphology of dendritic spines, and therefore its use in IBC.

The molecular mechanism of storing memory would be appropriately IBC relevant. An overall mechanism of interest is the strengthening of synapses by creating proteins of the likes of Lypla, Cam Kinase, et al. The creation of those proteins happens by the relevant RNA that is otherwise blocked (from creating those proteins) with wrapping proteins, which are degenerated or broken down when new signals arrive freeing up the RNA to create the strengthening proteins. The use of this and allied mechanisms would improve IBC.

The absence of the chloride channel CIC-2 in nerve cells has been found to make them more excitable, hence in principle more capable of either transmitting or inhibiting information (the latter when such neurons are “inhibitory”), rendering that a possible means of controlling IBC.

The extent of glutamate released and reabsorbed (post signaling) would be a factor to be considered on how effectively neurons communicate with each other in the IBC context.

Advanced non-rigid ultra-thin electrodes in the 100 to 500 micrometer thickness range that are better able to mould to the human brain and transmit information much better than common rigid electrodes have already been developed, and these would if necessary assist in IBC.

Special DNA segments, called “enhancer regions”, are known to cause the release of specific RNA called “enhancer RNA” (eRNA) to release proteins that would augment the creation of synapses across neurons post stimulation, thereby enabling learning, making this of relevance to enhancing IBC.

The significance of “mirror neurons” that fire both when a human performs a task and the human watches another human perform a task, would be considered in the IBC context. The limbic striatum has been determined to be significant to implicit learning, and hence would be given due focus vis-à-vis IBC.

The effects of the hunger hormone ghrelin, produced in the stomach and pancreas, on memory and learning (by influencing the hippocampus) is of some relevance to IBC. Ghrelin is known to augment dendritic spine formation in the hippocampus and (thereby and/or otherwise) affect long term potentiation (LTP). IBC learning could be expedited and rendered more effective with the optimal administration of ghrelin.

The effect of neuron-to-glial-cell communication is pertinent, and would be specifically analyzed, in the context of enhancing neuron-to-neuron communication from the IBC perspective. The mechanisms of such neuron-to-glial-cell communication would include but not be limited to the neuron's synaptic and non-synaptic releases of specialized signaling chemicals, as well as other chemicals (e.g. neurotransmitters, ions, etc released into the glia) that ultimately boost neuron-to-neuron communication. Of particular interest would include, but not be limited to, the use of the calcium ions in the 3-way communication between glial cells and post- and pre-synaptic neurons. The glial cells of the greatest significance in this context are the oligodendrocytes and astrocytes of the CNS. Astrocytes are known to be able to regulate synaptic plasticity, not only as small groups on a single synapse but as larger communicating groups of astrocytes that combined affect a network of neurons. Astrocytes are known to be able to generate Ca²⁺ signaling in response to neuronal activation, and its activity can be mediated by suitable agonists and antagonists, among others, thereby enabling an element of synaptic transmission control for use in IBC. Of secondary significance would be the ependymal cells and radial glia of the CNS. Schwann cells of the PNS, and to some extent even the role of microglia would be of some significance. Of added pertinence would be the role of P2X- and P2Y purinoceptors in the communication between glial and neuron cells using nucleotides, where gliotransmitter ATP plays a role. In addition, of interest would be determining if ephrin ligands and eph receptors could be optimally utilized towards neuron-glia communication, for enhancing IBC.

Fluorescence Resonance Energy Transfer (FRET) and/or allied technologies would be considered for IBC towards live observation of chemical synapse operation where necessary.

Protocadherin gamma-C3 (PCDHGC3), Protocadherin gamma-A11 (PCDHGA11), Protocadherin gamma-B7 (PCDHGB7), Protocadherin gamma-A12 (PCDHGA12), Protocadherin beta-13 (PCDHB13). Protocadherin beta-9 (PCDHB9), Protocadherin beta-16 (PCDHB16). Protocadherin beta-11 (PCDHB11), Protocadherin beta-14 (PCDHB14), Protocadherin beta-10 (PCDHB10). Protocadherin beta-3 (PCDHB3), Protocadherin beta-2 (PCDHB2), Protocadherin beta-4 (PCDHB4), Protocadherin beta-15 (PCDHB15). Protocadherin beta-7 (PCDHB7), Protocadherin beta-12 (PCDHB12), Protocadherin beta-5 (PCDHB5) would be investigated for relevance to neuron-to-neuron communication for IBC.

The effect of L-serine and other amino acids on aspects of neuronal growth, hence neuronal communication, would be IBC pertinent.

The use of light sensitive ion channels called Channelrhodopsins (Chr) towards selectively switching off areas of the brain as part of discerning activation for data transmission would be looked into in the IBC context. ChR2 could be among the more promising in that context, with the other options being ChR1 and VChR1—all of them being sensitive to the cations K⁺, Na⁺, H^{+ and Ca}⁺⁺. Similarly haldosporin (NpHR) is a light sensitive ion channel that is sensitive to only anion (i.e. negative) chloride ions. ChR2 is sensitive to blue light (maximum absorption at wavelength 480 nm), while VChR1 is sensitive to yellow and could potentially trigger neurons around 580 nm. The use of the Arch and Mac genes, to switch off neurons when exposed to yellow and blue lights respectively, is another effective option, whereby those genes could be loaded onto viruses and inserted into neurons without affecting their operations, and when those neurons are exposed to blue or yellow light energy producing proteins in the cells are inhibited, resulting in the overall temporary deactivation of the neuron, constituting an IBC managing mechanism.

### The Reversibility and Multi-Candidature Capabilities of Self Identity Transfer

With the feasibility of IBC already logically established across the above and earlier sections of this section, this section is not directly on IBC's feasibility but instead on some additional highly significant features of the invention, in the context of IBC.

Here's the gist, followed by summary elaborations:

Self identity transfer would be reversible, as long as the first human's body is still alive. That reversibility would be possible both at the end of Step 2 (when the self identity of the first human would be provisionally present in both brains (i.e. when the entire “transfer” of self identity (i.e. rendering self identity present in the second brain along with in the first brain and then removing it from the first brain) will have yet not happened, only “transfer” in the sense of its provisional presence in both brains will have happened)) or if elected at the end of Step 3 (when the self identity of the first human would be present in the second brain only).

The memory data that would be copied to the second brain as part, of Step 2 could be separately copied to the brains of multiple people as a separate set of Steps 1 & 2 for each, without undertaking Step 3 for any of them. The first person's self identity would remain not fully transferred (i.e. not rendered first present in the second brain along with in the first brain and then removed from the first brain) until Step 3 is completed with any one of them, so there'd be no loss of self identity from the first brain regardless of undertaking Step 2 for several different memory-recipient brains. Because Step 3 wouldn't mandatorily need to be undertaken immediately after Step 2, this would enable a choice of candidates to transfer to over Step 3 at the later time when the decision to transfer self identity over Step 3 is taken. That choice would be meaningful because external circumstances might render some of the memory recipient brains (i.e. those for which Step 1 and Step 2 will have been done) subsequently unavailable/unsuitable when Step 3 is decided.

As may be expressly inferred from the IBC details in this section, the actual IBC link would not need to be over physical wires, and could be entirely over wireless if so chosen.

It may be importantly noted that IBC proceedings in the invention would first be undertaken to comprehensively complete memory transfer activities (i.e. undertaken primarily as part of Step 2 of the four steps of the invention)—a process that would take time and therefore would need to be commenced adequately in advance of the actual physical body-death of the first human and (as an added procedural bonus) wouldn't require continuous IBC connectivity (i.e. it could be done in several separate sequential IBC sessions/phases) since memory data once transferred and stored in the second brain wouldn't ordinarily need to be retransferred—and only the core self-identity “transfer” (i.e. Step 3 of the invention) (that would not necessitate much memory data (re)transfer, optionally other than for some new latest and/or earlier memory data if so elected) would be left as the final, separate, IBC activity to be undertaken within a short span of time typically not long before the actual physical body-death of the first human. Because this step would be separate, there would be no need for the two human brains to remain IBC (re)connected until just before the point of that quick self identify transfer, thereby allowing the humans complete freedom to go about their own separate lives following conclusion of the memory data transfer of Step 2, until just before the decision is taken for self identity to be transferred from the first human to the second. Appropriate of the already previously created artificial and natural synapses and other hardware connectivity “ports” created for IBC would then be quickly reused to reestablish IBC towards that final step, to transfer self identity. Also, importantly, because largely no new artificial/natural synapse or other “port” hardware would need to be created for Step 3 of the invention, and since that final act (per Step 3) of self identity “transfer” would essentially constitute enabling access to already-transferred-and-stored-memory at the second brain, temporary simultaneous presence in both brains of the intended-to-be transferred self identity (i.e. the logical unification of the two logical centers of consciousness in the two brains into a single one), and then “switching off” the first brain's component of self identity, the process of self identity transfer would be reversible as long as the first body is physically alive.

Furthermore, multiple candidates for self identity transfer could be readied in advance (by completing extensive memory transfer to each) to reasonably ensure the availability of at least, one candidate when self identity transfer is finally elected.

These reversibility and multi-candidature aspects of self identity transfer would be of enormously significant practical benefit, as they would allow anticipatory full transfer (or simultaneous presence in two brains in preparation for full transfer) of self identity (i.e. in reasonable anticipation of possible physical death of the first body, for example if the first human is at the end stages of any terminal illness and therefore the body is likely to die in the imminent/near future, or if that first human is on an airplane that develops critical engine failure en route and appears unlikely to be able to land safely, so self identity is quickly simultaneously established in the second brain (i.e. simultaneous to that self identity also remaining in that human's own brain) over wireless IBC (to prevent disease transmission in the first example (of the terminally ill person), and wireless IBC would anyway be logistically mandatory in the second example (of the airline passenger)) for any one of multiple candidates who is healthy and who is then safely on the ground or elsewhere not on that particular airplane, and that self identity is either deliberately not fully transferred—if ever—until at the last, moment to maximally wait to complete Step 3 to be sure on the likelihood of any impending physical body-death or transferred anyway without waiting further, completing Step 3), and then if so elected reverse the process later while still in the simultaneous-self-identity-in-two-brains stage (i.e. with Step 3 in progress but not completed) or even at a post transfer stage (i.e. with Step 3 completed) should the anticipated physical death of the body doesn't actually happen (e.g. if the person in the above first example somehow recovers from that terminal illness, or if the airplane in the above second example is able to land safely). As indicated above, even if self identity is fully transferred, as long as the first body is still alive self identity can be transferred back in a mechanism similar to but significantly less cumbersome than how the identity was transferred out in the first place.

### The Minimalism Option of Self Identity Transfer

An optional, overall much faster but less comprehensive, form of perpetual persistence of life with this invention using IBC would be to in large part forego the extensive non-essential memory transfer component, and undertake primarily just the self identity transfer component (inclusive of the fundamental, core essential memory absolutely mandatory for self identity transfer) over IBC. Post self identity transfer the individual would still be consciously able to recognize herself/himself in the second brain and also retain the minimal core sub-/unconscious memory (e.g. behavioral) traits and “adjust” into a modified, yet always acceptable (see the final two items of “Feasibility of invention” earlier in this section), rendition of herself/himself with selections of the already preexisting memory data in the second brain, albeit that second brain would lack most of the non-essential memory data that was present in the first brain.

### CONCLUSION

IBC is logically reasonably feasible substantially exceeding the thresholds necessary for medically perpetual persistence of essential human life. That feasibility can be practically demonstrated by implementing the enablement principles outlined in the original patent application and in this section, in an R & D process that would take its due resources and time.

### DESCRIPTIVE LEGENDS ON FIGURES

FIG. 1

Towards creating the integrated, augmented, single, logical center of consciousness, each of the 7 brains within the dashed box has in this example been shown to be linked only to Mary-I's brain. However, in reality some links could also be chosen to be among themselves (i.e. contained within the dashed box) and not all need to be directly linked to Mary-I. Ultimately, upon delinking them all, ordinarily at most one brain could have Mary-II-B, and the rest Mary-II-A.

FIG. 2

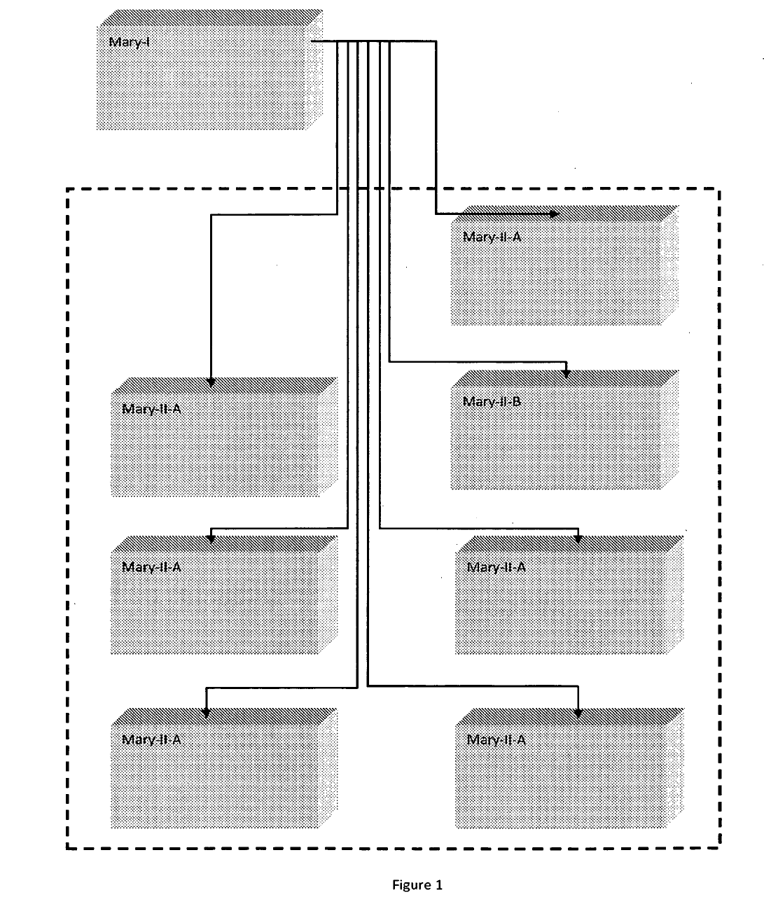

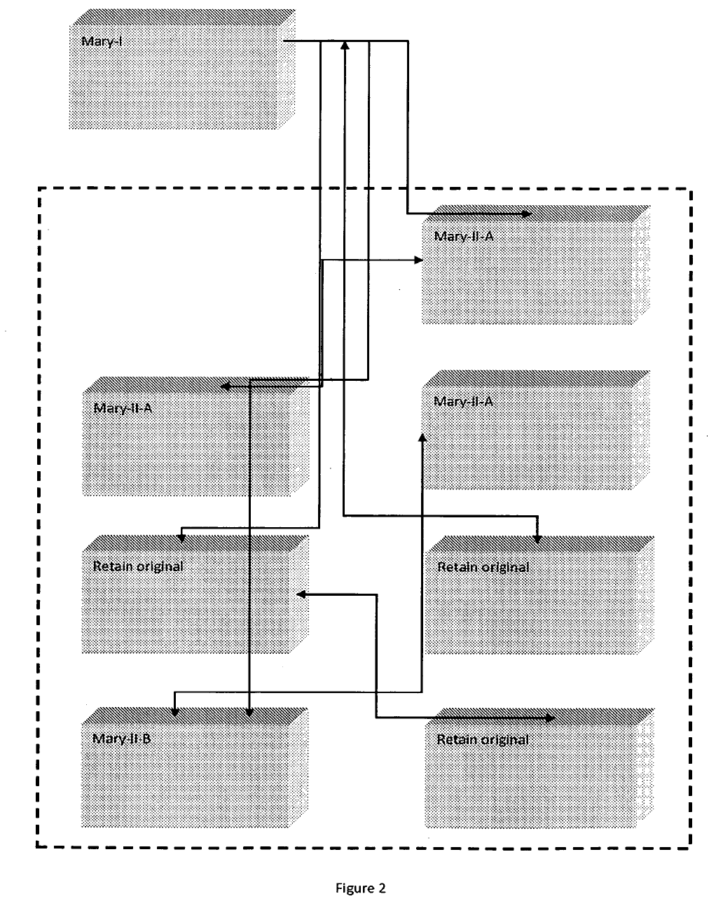

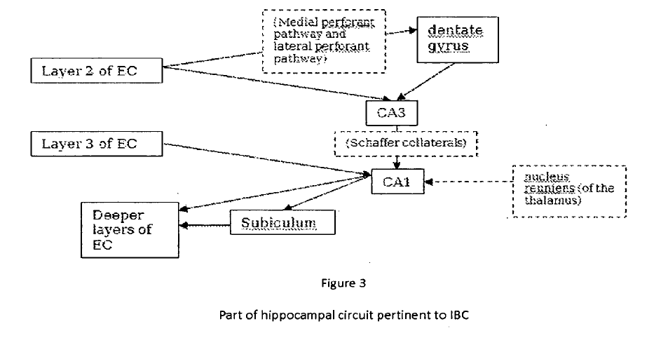

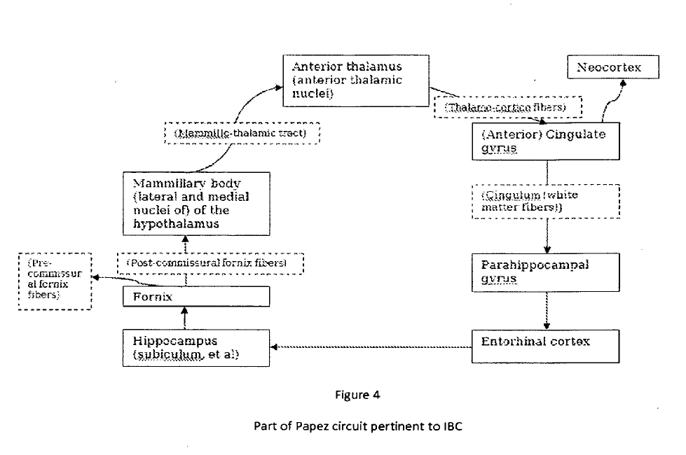

In this example, the 7 brains in the dashed box are selectively directionally interlinked among themselves and with Mary-I's brain, towards the formation of the integrated, single, logical, augmented center of consciousness. Note that the directions indicate information transport (hence consciousness transport prospect) direction. Again, upon delinking, ordinarily at most ONE Mary-II-B brain may be formed, and at the rest at most Mary-II-A. Some can choose to retain solely their original identities.

### Legal

This section is to be deemed an appropriate extension of the original patent application cited in the section, and presents the inventor (and applicant's own conclusions on how to approach and implement IBC. All conclusions on or related to IBC, and/or otherwise on the remainder of the cited invention “Medically perpetual persistence of essential human life”, presented in this section are entirely the inventor (and applicant's own, regardless of whether or not the inventor (and applicant) has expressly mentioned such in this section. A primary intent of this section is to provide supportive technical logical evidence proving the feasibility of IBC detailed in the original patent application (and therefore also detailed in this section), on which (IBC and allied technology aspects) the inventor (and applicant) effectually owns and/or would reasonably own intellectual property rights pursuant to the original patent application. Some aspects of the core technical approaches to IBC outlined in this section surmise the inventor (and applicant's technical inferences premised on or otherwise lawfully conceptually derived from, where applicable, relevant Limited information already available in the public domain—any intellectual property rights (i.e. if any at all. and where applicable if so) on which (aforementioned already publicly available information) is/are or would be owned by the respective due lawful entity/-ies other than the inventor (and applicant), and as (to be) determined by law the inventor (and applicant) would own intellectual property rights on appropriate of such derivative techniques listed in this section. Because it is outside the scope of this section to conduct extensive legal research and deliberation to fairly determine who (other than the inventor (and applicant)), if any at all. own(s) or would own or should own intellectual property rights on the aforesaid publicly available limited information, this section refrains from attempting to credit such rights, and as aforementioned expressly leaves it to the courts and/or other government branches to determine and for the readers of this section to look up the appropriate rights ownership databases (on, but not limited to, patent(s), trademark(s); service mark(s), copyright(s), etc.) as and where applicable. Any and all item(s) that might be listed in this section, or/and in the remainder of the entire patent application, on which intellectual property right(s) (including, but not limited to patent(s), trademark(s), service mark(s), copyright(s), etc.) is/are currently already owned is/are just that—its/their current intellectual property ownership is as currently listed in the appropriate lawful official database(s) on such intellectual property ownership. While every reasonable effort has been taken to prevent technical errors while keying in (i.e. typing) this section, in the highly unlikely event that one or more technical error(s) while keying in (i.e. typing) has/have encroached into the section such keying in (i.e. typing) error(s) is/are to all be correctly interpreted by the reader, and the already affirmed validity of the invention remains entirely unaffected under all circumstances regardless of any and all such keying in (i.e. typing) error(s).

What is claimed is:

1. A single, unified, prophetic invention comprising of a method of enabling indefinite extension to human lifespan, comprising of the following four (4) steps, as defined in two separate renditions (i.e. a fundamental rendition and an advanced rendition) of the same single, unified invention:
I Fundamental rendition of this single claim of this single, unified invention—
Step A of I
The brain of the individual (the latter labeled “Mary-I” for purposes of this claim) whose lifespan is sought to be extended indefinitely is linked to directly communicate with the brain of another individual (the latter labeled “Mary-II-B” for purposes of this claim) using what in this patent application is termed “interbrain communication (IBC)” and described (in this patent application), where (to define IBC) each and all of the following aspects of the definition of IBC would be specifically and highly significant:
(a) IBC would be an optimized functional and physical (i.e. anatomical) integrative process whereby direct communication is achieved between the two brains that (communication) would be primarily between selections of anatomically equivalent (herein often termed “IBC relevant”) locations of the two brains whereby such (locations) would primarily but not limited to pertain to appropriate memory and consciousness functions, the selection of which (anatomically equivalent locations) would be suitably optimized or modified based on functional equivalence; Hyper-precise identification of IBC relevant locations in the two brains would not be necessary, reasonable precision in equivalence would suffice as with such reasonable precision in equivalence the brains would naturally filter out irrelevant signals (from and otherwise caused by the other brain) and naturally physiologically adjust to filter in relevant signals (from and otherwise caused by the other brain); The essential approach would be a 1:1 equivalence-component-mapped interconnectivity simultaneously for all IBC relevant components of count “n” in the two brains, where n<N, “N” being the count of all (i.e. inclusive of non-IBC relevant) corresponding components in each of the two brains; additional considerations are to simultaneously, spanning all n components, have grouped m:m, m:M or M:m interconnectivity, where m<M<n<N with the groups being functionally (and therefore and otherwise physiologically and/or functionally) relevant, to enable greater efficiency yet ensuring that data integrity (i.e. including but not limited to quality in terms of relevance) would remain as good as in the 1:1 architecture;
(b) Dendritic spines would be an important consideration, because of their ability to efficiently change shape/size/count, hence optimal production and use of dendritic spines would be critical to optimizing IBC, to enable huge volumes of memory (and allied) data transfer fast without compromising the integrity of such data;
(c) The signals at inter-hemispheric neuronal pathways would be profoundly focused on with the objective of (to the maximum meaningful extent) directly accessing them for IBC towards easier access into the respective hemispheres; significant among such IBC relevant inter-hemispheric neuronal connections are the corpus callosum, the anterior commissure and the hippocampal commissure (also known as the commissure of fornix);
(d) Artificial synapse and/or natural synapse creation and/or enhancing techniques would be applied;
(e) A specific set of areas of the brains would be selected to prioritize the focus of IBC on, that primarily, but not limited to, comprises of one or more (typically simultaneously several/most) of the group (listed below comprising both superset components and their member (sub)components (and therefore and otherwise listed below to particularly not exclude direct and/or indirect repeat mentions of the same component(s)), for emphasis and appropriate (re)classification grouping):
prefrontal cortex, amygdala (cortical nucleus, central nucleus, medial nucleus, basolateral nucleus), hippocampus, precuneus and other areas of the prosencephalon; basal ganglia and the cerebellum; neocortex (neopallium) of the telencephalon and the thalamus, hypothalamus, sub thalamus and epithalamus of the diencephalon; the neopallium subcomponents frontal cortex (specifically including and necessarily not limited to its prefrontal cortex), parietal cortex, anterior cingulate and parts of another component of the telencephalon—the basal ganglia; sections of the limbic system, namely the thalamus (including but not limited to the centromedian nucleus (part of the intralaminar nucleus) also termed the centrum medianum (Cm-Pf/CM)), entorhinal cortex, limbic cortex, anterior thalamic nuclei, mamillary body, fornicate gyrus (inclusive of, but not limited to, its cingulate gyrus and the inferior parahippocampal gyrus), fornix, dentate gyrus, orbitofrontal cortex; parahippocampal gyrus with particular note to entorhinal and perihinal cortices; mammillary bodies; Papez circuit; orbitofrontal cortex; medial temporal lobe (MTL); caudate nucleus, putamen; subthalamic nuclei; primary motor cortex, pre-motor cortex, supplementary motor cortex; superior (and inferior) frontal gyrus; fusiform gyrus (including but not limited to FFA (Fusiform Face Area)); areas in temporal lobe (with particular note, but not limited, to Brodmann areas BA20, BA34); primary somatosensory cortex, posterior parietal cortex, postcentral gyrus; insular cortex; cingulate cortex (with particular note to subgenual area 25 (BA25), anterior cingulate, retrosplenial cortex (RSC); Brodmann areas 41, 42 and 22p; mesocortical-, mesolimbic- and nigrostriatal dopamine pathways; Nucleus Basalis of Meynert (NBM); temporal neocortex; tectum of mesencephalon; (metencephalon and myelencephalon of the rhombencephalon would overall be of low relevance), pons; neocerebellum;
(f) IBC would be established between the two brains at (or otherwise compatible/synchronized to) the appropriate signal wave frequencies (including but not limited to, where pertinent, in synchronized theta (4-7 Hz) and alpha (8-12 Hz) frequency ranges, given that those frequencies are deemed highly relevant to memory encoding and retrieval), strengths and durations—including but not limited to as a variable sequence of “signal bursts” of electromagnetic and/or electrical signals, while noting that IBC would (also) be appropriately directed to the respective input/output neural circuit areas—i.e. the output of a component of one brain fed to the input of the component in the other brain that corresponds to the component in that same (former) brain that would ordinarily receive that input (instead of and/or in parallel addition to naturally feeding that output to the equivalent component in the same (former) brain itself as well) and vice versa; the beta (12-30 Hz) and gamma (>30 Hz) frequencies would also be relevant for IBC, both when those are the base frequencies of the corresponding brainwaves at those locations as well as otherwise for augmenting IBC efficiency and quality; a plurality of techniques of signal synchronization would be effected; two brains will not necessarily operate in the exact same frequencies and phases even at considerably precisely identified (anatomically and functionally integrated and optimized) equivalent sites: hence optimal frequency-phase-intensity-duration combination (s) corresponding to a plurality of functionally equivalent sites across the two brains would be identified specific to those two brains;
(g) A plurality of categories of event related potential (ERP) would be used to identify the IBC relevant functionally equivalent locations in the two brains, including but not limited to considerations of one or more of N100, N400, P300 (P3), SPS (P600), ELAN (early left anterior negativity), late positive potential (LPP), ERP P200 (P2), N270, N430, P3a, P3b; to identify the separate component ERP signals a plurality of signal analysis techniques would be applied;
(h) A plurality of techniques would be applied to optimize IBC signals; such techniques would comprise of (i.e. include but not be limited to) considerations of one or more of CREB (cAMP response element binding) protein, NR3A NMDAR(NMDA (N-Methyl-D-Aspartate) receptor)s, PolyADP-ribosylation, D1 receptors, AMPA receptors, CAM kinase II, MEF2C (MADS box transcription enhancer factor 2, polypeptide C (also known as Myocyte-specific enhancer factor 2C)); neuropeptides towards augmented neuronal interconnectivity;
(i) Molecular mechanisms of storing memory would be an important consideration;
(j) Neuron-to-glial-cell communication would be highly pertinent;
(k) A plurality of mechanisms enhancing neuron-to-neuron communication would be applied;
(l) A plurality of innovations and allied techniques, including but not limited to those relevant to (b) through (k), aimed at effecting and/or enhancing the fundamental objectives cited in aspect (a) would be applied;
Step B of I
Using the IBC channel established in Step A of I, Mary-I learns to use the brain of Mary-II-B, towards forming (together with Mary-I's own brain) an integrated, (virtual) single, augmented, logical brain containing an integrated, augmented, logical center of consciousness;
Step C of I
Using the IBC channel established in Step A of I, Mary-I “relocates” to Mary-II-B's brain and then terminates the IBC channel established in Step A of I, thereby Mary-I then physically disassociates from Mary-I's original own brain;
Step D of I
Complete cycles, comprising of each of Step A of I, Step B of I and Step C of I in that sequence, are repeated sequentially for as long as MaryI's lifespan is elected to be extended, where, in each new cycle, the human designated Mary-II-B in the immediately previous cycle will be designated Mary-I (in that new cycle) and a new Mary-II-B chosen (in that new cycle);
II Advanced rendition of this same, single claim of this same, single, unified invention, (this advanced rendition of that claim) constituting an encompassing superset of the aforementioned fundamental rendition of this same, single, claim—
Step A of II
The brain of the individual (the latter labeled “Mary-I” for purposes of this claim) whose lifespan is sought to be extended indefinitely is linked to directly communicate with the brain of just one other individual (the latter labeled “Mary-II-B” for purposes of this claim) or a plurality of individuals (each (or a group) of the latter to be labeled in Step C of II either “Mary-II-A” or “Mary-II-B” (the former label (“Mary-II-A”) if in Step C of II Mary-I does NOT select that individual (or group of individuals) to “relocate” to, and the latter label (“Mary-II-B”) if in Step C of II Mary-I DOES select that individual (or group of individuals) to “relocate” to)) using, mutatis mutandis, what in this patent application is termed “interbrain communication (IBC)” and described (in this patent application) and defined in Step A of I;
Step B of II
Using the IBC channel established in Step A of II, Mary-I learns to use other brain(s), towards forming one integrated, augmented brain (or a combination of communicating integrated, augmented brain systems) containing one integrated, augmented, logical center of consciousness (or a combination of communicating, integrated, augmented, logical centers of consciousness, only one of which would have authority over the set of participating brains in that particular augmented, integrated, logical center of consciousness, at any one time); Each integrated, augmented, logical center of consciousness would comprise of in whole or in (typically the most) part, the logical center of consciousness (hence include an appropriate fraction of the consciousness component therein of the associated brain) of each individual constituent brain, and (typically) an additional minor consciousness component (that may preferably be reduced or eliminated for the integrated, augmented consciousness to maintain acceptable fidelity to the logical integration of the original set of consciousnesses) originally associated with none of the constituent brains; Each integrated, augmented, logical center of consciousness will be physically located across one or up to all of the individual single original centers of consciousness in each constituent brains—preferably (for upcoming (per Step C of II) Mary-II-B's logistical convenience) in exactly one of the constituent brains' centers of consciousness—and only one integrated, augmented, logical center of consciousness may control a constituent brain at any one point of time, even though that brain may be part of multiple separate integrated, augmented, logical centers of consciousness; As part of that learning Mary-I would consciously, subconsciously and unconsciously adequately recognize herself (inclusive of all, most or an acceptable extent of her key original memories) using the (and each, when multiple) integrated, augmented, logical center(s) of consciousness—hence the logical center(s) of consciousness in the other brain(s); That recognition would alternatively be, at Mary-I's conscious/subconscious/unconscious initiative/will, using some/all the centers of consciousness (i.e. at will, including her original own and the other(s) and separately using only the other(s) and not her original own); As indicated at the beginning of Step B of II, at the end of this step will be formed one integrated, augmented brain (or a combination of communicating integrated, augmented brain systems) including Mary-I's brain, containing one integrated, augmented, logical center of consciousness (or a combination of communicating, integrated, augmented, logical centers of consciousness, only one of which would have authority over the set of participating brains in that particular augmented, integrated, logical center of consciousness, at any one time);
Step C of II
Using the IBC channel established in Step A of II, Mary-I chooses a logical center of consciousness (if multiple individual logical centers of consciousness have been created in Step B of II) or individual (in whose brain the single final logical center of consciousness would consequently become located as subsequently defined herein), “relocates” to it, logically disassociates from Mary-I's original brain (i.e. from the original center of consciousness of Mary-I) (Note—while a physical disassociation from Mary-I's brain is not mandatory, that will typically ultimately follow), and therefore manifests Mary-II-B within that chosen logical center of consciousness; The logical center of consciousness would ideally be located physically in just one of those other (i.e. non-Mary-I) brains—hence in the center of consciousness therein that one brain—for enhanced logistical convenience of Mary-II-B (but could in principle be located in more than one of those brains as well, as long as they're logically linked forming one integrated, augmented, center of consciousness); At the end of that transfer Mary-I's brain will be logically, and in practice often physically, disassociated from the integrated, augmented, logical center of consciousness that Mary-II-B has relocated to;
Step D of II
Complete cycles, comprising of each of Step A of II, Step B of II and Step C of II in that sequence, are repeated sequentially for as long as Mary-I's lifespan is elected to be extended, where, in each new cycle, the human (or group of humans) designated Mary-II-B in the immediately previous cycle will be designated Mary-I (in that new cycle) and a new Mary-II-B chosen (in that new cycle);
Notes—
Note #1 (applies to each of I and II)
As indicated elsewhere. Steps A, B, C and D are each part of a single, unified invention, constituting the only feasible mechanism of indefinite extension to human lifespan; However, in order to try to prevent selective/limited violations by third party/-ies of intellectual property rights (of this applicant/inventor (or any future assignee(s)) on this invention) vis-a-vis one/more of the plurality of aspects of the invention, it is hereby clarified that also included in the scope of this one (1) single claim of this patent application, in lawful reasonableness, are the following—
i. All reasonable morphs/variations/renditions/forms/flavors of the sequence of three steps Step A, Step B and Step C, with or without Step D;
ii. All reasonable morphs/variations/renditions/forms/flavors of Step B and Step C by themselves or together in any sequence, whether or not preceded by Step A and/or succeeded by Step D;
iii. All reasonable morphs/variations/renditions/forms/flavors of Step A by itself to the extent of IBC;
iv. All reasonable morphs/variations/renditions/forms/flavors of Step A by itself to the extent that it is relevant to indefinite extension of human lifespan (when such Step A is used as the first step preceding all reasonable morphs/variations/renditions/forms/flavors of Step B and/or Step C with and/or without Step D);
v. All reasonable morphs/variations/renditions/forms/flavors of the single unified invention, hence this one (1) single claim, as applied, mutatis mutandis, to primates and other non-human living beings;
Note #2 (applies to each of I and II)
If any sub-component of the plurality of technologies to be used in IBC (and therefore and otherwise to be used in Step A, and/or in Step B, and/or in Step C and/or in Step D) is (then, or otherwise) already patented (that latter is or would be valid (i.e. not expired, withdrawn, etc)) by other than this inventor/applicant (and/or any future assignee(s) on this invention) then the scope of this claim only when such technology/technologies is/are used would be, in lawful reasonableness, as new use and/or improvement use;
Note #3 (applies to I, hence applies mutatis mutandis to II)
Aspects (b) through (k) of the definition of IBC in Step A of I are to effect and/or enhance, but not to narrow down, the fundamental objectives cited in aspect (a) of Step A of I; Hence aspects (a) and (l) together constitute the primary aspects of, and represent the breadth and depth of the scope of, the definition of IBC; Yet the fact that (b) through (k) (along with (a) and (l)) have been specifically listed in Step A of I as each being specifically and highly significant towards the definition of IBC would aid in identifying, in certain circumstances, the precise boundaries of the aforesaid breadth and depth of the scope of the definition of IBC in a scope delimiting—hence precise scope identifying—perspective; In other words, it would be easier, in applicable circumstances, to identify the boundaries of the breadth and depth of the scope of IBC when taking into account that each of the aspects (a) through (l) (i.e. inclusive of aspects (b) through (k)) is specifically and highly significant than the fact that just (a) and (l) together define the breadth and depth of IBC's scope;
Note #4 (applies to I, hence applies mutatis mutandis to II)
As should be evident to any technically competent individual skilled in the sciences and technologies relevant to this invention, the technical specifics represented in aspects (b) through (k) of the definition of IBC in Step A of I have either been expressly or reasonably implicitly already included in the original patent application (PCT International Application No. PCT/IB2009/052355 filed with RO/IB on 3 Jun. 2009) and in its priority document incorporated therein that original application by reference in its entirety (USPTO Provisional Patent Application No. 61/058,253 filed on 3 Jun. 2008); Nevertheless, such specifics were (again) expressly (re)filed with RO/IB on 28 Aug. 2010 and registered by RO/IB on 30 Aug. 2010; In order to try to avoid any confusion by whomever reviews this claim, particulars of the invention's description pertinent to such (re)filed specifics have been expressly included in this patent application; Accordingly, (and directing attention to Note #3), because, in actual effect, including but not limited to in as much as the lawful scope of this invention (and hence of this claim), nothing substantively new has been added to the specifications of the invention in this patent application than were already expressly or reasonably implicitly included in the aforesaid original patent application (PCT International Application No. PCT/IB2009/052355 filed with RO/IB on 3 Jun. 2009) and in the latter's priority document (USPTO Provisional Patent Application No. 61/058,253 filed on 3 Jun. 2008), this patent application is lawfully no less eligible to be processed either as a national phase PCT application or a continuation (or equivalent) application than as a continuation-in-part (or equivalent) application, appropriate to the laws of the respective nations.
